(19) United States
(12) Patent Application Publication
(10) Pub. No.: US 2021/0353743 A1
(43) Pub. Date: Nov. 18, 2021

(54) BIOMATERIAL-BASED COVID-19 VACCINE

(71) Applicant: Northeastern University Center for Research Innovation, Northeastern Univ., Boston, MA (US)

(72) Inventors: Sidi A. Bencherif, Boston, MA (US); Thibault Colombani, Boston, MA (US)

(21) Appl. No.: 17/322,054

(22) Filed: May 17, 2021

Related U.S. Application Data
(60) Provisional application No. 63/071,736, filed on Aug. 28, 2020.
(60) Provisional application No. 63/025,401, filed on May 15, 2020.

(51) Int. Cl. A61K 39/215; A61K 38/44; A61K 45/06

(52) U.S. Cl. CPC A61K 39/215; A61K 2039/55561; A61K 45/06; A61K 38/44

(57) ABSTRACT

Disclosed are compositions and methods for preventing an infection, such as COVID-19 infection, comprising administering to the subject an oxygen-generating cryogel, wherein the oxygen-generating cryogel comprises an antigen, a chemoattractant for immune cells, an adjuvant, such as toll-like receptor 9 (TLR9) ligand, and an oxygen-producing compound. Described herein are methods of modulating the immune system in a subject, comprising administering an effective amount of the vaccine described herein to the subject, thereby modulating the immune system in the subject.

### RELATED APPLICATIONS

This application claims the benefit of priority to U.S. Provisional Patent Application Ser. No. 63/025,401, filed May 15, 2020; and U.S. Provisional Patent Application Ser. No. 63/071,736, filed Aug. 28, 2020.

### GOVERNMENT SUPPORT

This invention was made with government support under Grant Number 1847843 awarded by the National Science Foundation. The government has certain rights in the invention.

### BACKGROUND

Severe acute respiratory syndrome coronavirus 2 (SARS-CoV-2) has caused a global pandemic with over 30 million cases and nearly 1 million deaths as of September 2020 with no indications of slowing down. In response, several strategies are currently under rapid investigation, including treatments (e.g., antivirals, antibodies, anti-inflammatory, and immunomodulatory factors), and prophylactic vaccines (e.g., nucleic acid-based, protein subunit-based, recombinant viral vector-based, inactive or attenuated viral-based, virus-like particles). Yet, only vaccines have the potential to confer global immunity. The stakes are high: The alternative is natural herd immunity, requiring several waves of infection over the next few years, a period characterized by high mortality, economic uncertainty, and a perturbed way of life. Although two vaccine candidates have been approved by the Food and Drug Administration (FDA), it is unclear if they will alter the course of the pandemic and confer long term immunity. Therefore, there is a critical need to continue driving novel vaccination platforms into the clinic until SARS-CoV-2 is quelled, as well as to prepare for future pandemics.

### SUMMARY

In one aspect, described herein is a vaccine, comprising an oxygen-generating cryogel, wherein the oxygen-generating cryogel comprises an antigen, a chemoattractant/stimulating factor for immune cells, an adjuvant, and an oxygen-producing compound. Numerous embodiments are further provided that can be applied to any aspect of the present invention described herein. For example, in some embodiments, the antigen is a polysaccharide, a lipid, or a nucleic acid, or preferably a peptide. In some embodiments, the peptide is derived from a pathogen. The antigen may be a live, attenuated, or inactivated pathogen. In some embodiments, the peptide is derived from a viral S or N protein. In some embodiments, the pathogen is a virus such as a coronavirus, varicella-zoster virus, hepatovirus A, hepatitis B virus, hepatitis C virus, human papillomavirus, influenza virus, measles virus, marburg virus, rabies lyssavirus, variola virus, dengue virus, hantavirus, ebola virus, human papillomavirus, mumps virus, rubella virus, poliovirus, or rotavirus. In some embodiments, the coronavirus is selected from SARS-CoV, MERS-CoV, HCoV, HKU1, and SARS-CoV-2. In some embodiments, the antigen is a nucleocapsid protein, or a receptor-binding domain. In some embodiments, the pathogen is a bacterium, such as corynebacterium diphtheria, bordetella pertussis, clostridium tetani, haemophilus influenza, neisseria meningitides, streptococcus pyogenes, neisseria gonorrhoeae, mycobacterium tuberculosis, klebsiella pneumoniae, acinetobacter baumannii, streptococcus pneumonia, clostridium perfringens, pseudomonas aeruginosa, clostridium difficile, staphylococcus, methicillin-resistant staphylococcus aureus, or escherichia coli. In some embodiments, the pathogen is fungi, protozoa, worms, or parasites. In some embodiments, the pathogen is an infectious protein, such as a prion.

In some embodiments, the chemoattractant is granulocyte macrophage colony stimulating factor (GM-CSF), Flt3L, CCL-19, CCL-20, CCL-21, N-formyl peptide, ffactalkine, monocyte chemotactic protein-1, and MIP-3a. In some embodiments, the adjuvant is a toll-like receptor 9 (TLR9) ligand. In some embodiments, the TLR9 ligand comprises a cytosine-guanosine oligonucleotide (CpG-ODN). In some embodiments, the CpG-ODN belongs to Class A (Type D), Class B (Type K) and Class C. In some embodiments, the CpG-ODN is CpG-ODN 1585, CpG-ODN 1668, CpG-ODN 1826, CpG-ODN 2006, CpG-ODN 2007, CpG-ODN BW006, CpG-ODN D-SL01, CpG-ODN D-SL03, CpG-ODN 2395, CpG-ODN M362, CpG-ODN 2006-G5, CpG-ODN 2216, CpG-ODN 2336, CpG-ODN 2395, or CpG-ODN M362. In some embodiments, the TLR9 ligand is cavrotolimod, CMP-001, CpG-28, EnanDIM, amplivax, IMO-2125, lefitolimod, CpG-8954, or SD-101. In some embodiments, the adjuvant is a lipopeptide, glycolipid, proteolipid, virus, lipopolysaccharide, protein, mycoplasma, bacteria, fungi, peptide, polysaccharide, small synthetic molecule, toxoplasma gondii, oil, or inorganic molecule. In some embodiments, the virus is double-stranded RNA, poly I:C, single-stranded RNA. In some embodiments, the lipopolysaccharide is monophosphoryl-lipid A. In some embodiments, the protein is heat shock protein or Profilin. In some embodiments, the mycoplasma is multiple diacyl lipopeptide. In some embodiments, the bacteria is bacterial flagellin, lipoteichoic acid, multiple triacyl lipopeptides, or bacterial ribosomal RNA sequence “CGGAAAGACC”. In some embodiments, the fungi is zymosan (Beta-glucan). In some embodiments, the polysaccharide is a hyaluronic acid fragment, heparan sulfate fragment. In some embodiments, the small synthetic molecule is imidazoquinoline, resiquimod, bropirimine, or opioid drug. In some embodiments, the oil is a paraffin oil, propolis, or adjuvant 65. In some embodiments, the inorganic molecule is an aluminum salt.

In some embodiments, the oxygen-producing compound is a peroxide, an oxide, a percarbonate or a fluorinated compound. In some embodiments, the oxygen-producing compound is CaO₂, (Na₂CO₃)2.1.5H₂O₂, MgO₂, encapsulated H₂O₂/Polyvinylpyrrolidone, magnesium peroxide, hydrogen peroxide, manganese dioxide, perfluorochemicals, zinc oxide, or sodium percarbonate. In some embodiments, the oxygen-generating cryogel further comprises a catalase. In some embodiments, the catalase is an unmodified catalase or a catalase derivative. In some embodiments, the catalase derivative is acrylate-PEG-catalase (APC), acrylate-catalase, methacrylate-catalase, methacrylate-PEG-catalase, alkyne-modified catalase, or azido-catalase. In some embodiments, the vaccine is formulated with complete Freund's adjuvant (CFA) or is formulated with incomplete Freund's adjuvant (IFA). In some embodiments, the oxygen-generating cryogel comprises a naturally derived polymer, or synthetic polymer. In some embodiments, the naturally derived polymer is a polysaccharide, peptide, polynucleotide, or protein. In some embodiments, the polysaccharide is alginate, hyaluronic acid, or heparin. In some embodiments, the protein is gelatin, or collagen. In some embodiments, the polynucleotide is DNA or RNA. In some embodiments, the synthetic polymer is poly(ethylene glycol) (PEG), PEGylated glutaminase (PEG-PGA), PEG-poly(L-lactide; PLA), poly(2-hydroxyethyl methacrylate) (pHEMA), PAAm, or poly(N-isopropylacrylamide) (PNIPAAm).

In some embodiments, the oxygen-generating cryogel comprises a cryoprotectant or lyoprotectant selected from a sugar, polyol, amino acid, and b-cyclodextrin. In some embodiments, the sugar is sucrose, trehalose, mannitol, or cellobiose. In some embodiments, the amino acid is a histidine. In some embodiments, herein the oxygen-generating cryogel has a diameter of about 200 um to about 1000 um, has a diameter of about 1 mm to about 100 mm, or has a volume of about 100 um³ to about 500 mm³. In some embodiments, the oxygen-generating cryogel has a shape of spherical, cuboidal, ellipsoidal, or cylindrical. In some embodiments, the oxygen-generating cryogel reduces hypoxia in a biological tissue, reduces hypoxia such that oxygen tissue tension is higher than 3% oxygen, releases oxygen for at least about 4 h, at least about 16 h, at least about 24 h, or at least about 48 h, or at least about 96 h, releases oxygen for up to 48 h, or increases oxygen concentration in a biological tissue to physioxic or normoxic levels. In some embodiments, the vaccine triggers both Th1 and Th2 immune responses, promotes CD4+ and CD8+ T cell responses against the pathogen, promotes the production of Th1-biased and pro-inflammatory cytokines, or increases immunogenicity. In some embodiments, the increase in immunogenicity results in high level of binding and neutralization antibodies, results in stimulating B cells, results in high titer of antibodies against the antigen, or results in an increase in antibodies with high binding affinity. In another aspect, described herein is a method of modulating the immune system in a subject, comprising administering an effective amount of the vaccine described herein to the subject, thereby modulating the immune system in the subject.

In one aspect, described herein is a method of stimulating the immune system in a subject, comprising administering an effective amount of the vaccine described herein to the subject, thereby stimulating the immune system in the subject.

In one aspect, described herein is a method of inducing an immune response comprising administering to a subject in need thereof an effective amount of the vaccine described herein, thereby inducing an immune response in the subject.

In one aspect, described herein is a method of preventing an infection caused by a pathogen, comprising administering to a subject in need thereof an effective amount of the vaccine described herein to the subject. Numerous embodiments are further provided that can be applied to any aspect of the present invention described herein. For example, in some embodiments, the pathogen is a virus, such as a coronavirus, varicella-zoster virus, hepatovirus A, hepatitis B virus, hepatitis C virus, human papillomavirus, influenza virus, measles virus, marburg virus, rabies lyssavirus, variola virus, dengue virus, hantavirus, ebola virus, human papillomavirus, mumps virus, rubella virus, poliovirus, or rotavirus. In some embodiments, the coronavirus is selected from SARS-CoV, MERS-CoV, HCoV, HKU1, and SARS-CoV-2. In some embodiments, the pathogen is a bacterium, such as corynebacterium diphtheria, bordetella pertussis, clostridium tetani, haemophilus influenza, neisseria meningitides, streptococcus pyogenes, neisseria gonorrhoeae, mycobacterium tuberculosis, klebsiella pneumoniae, acinetobacter baumannii, streptococcus pneumonia, clostridium perfringens, pseudomonas aeruginosa, clostridium difficile, staphylococcus, methicillin-resistant staphylococcus aureus, or escherichia coli. In some embodiments, the vaccine is administered by injection. In some embodiments, the vaccine is administered by injection using a hypodermic needle, or by injection using a catheter. In some embodiments, the vaccine is administered intramuscularly or subcutaneously. In some embodiments, the subject is a mammal such as a human or a mouse.

### BRIEF DESCRIPTION OF THE DRAWINGS

FIGS. 1A-1D show that cryogel-based vaccines reinforce DC-mediated immune responses. FIG. 1A shows quantification of cellular hypoxia in cells recruited within Cryogels and O₂-Cryogels 24 h and 72 h post-subcutaneous injection in mice. FIG. 1B shows quantitative flow cytometric measurements of CD86^{High} and CD317^{High} dendritic cells (CD11c^{High}) in Cryogels or O₂-Cryogels in presence or absence of CpG-ODN 1826 in hypoxia and normoxia for 24 h (right panel). Values represent the mean ±standard error of the mean (SEM, n=5 per group). ***P<0.001. FIG. 1C shows overview of the process for fabrication and evaluation of cubiform cryogel and O₂-Cryogel vaccines. Step 1 involves freezing vaccine components, enabling crosslinking of solutes around ice crystals (step 2). Thawing results in an interconnected macroporous network with vaccine components encapsulated within the polymer network (step 3). Addition of calcium peroxide and catalase to the vaccine components before cryogelation produces oxygen-generating cryogel vaccine (O₂-Cryogel_{VAX}) capable of sustained production of oxygen. In step 4, cryogels are subcutaneously injected into mice for preclinical vaccine studies. FIG. 1D shows illustration describing a model for DC-enhanced cryogel-induced immunity. HAGM: hyaluronic acid glycidyl methacrylate; initiator: ammonium persulfate and tetramethylethylenediamine.

FIGS. 2A-2E show O₂-Cryogel_{VAX} induces robust binding and neutralizing antibody responses against SARS-CoV-2 in mice. FIG. 2A shows study timeline describing the vaccination regimen (BALB/c mice; n=5 per group) and the timing of the different sample collection and immuno-assay performed in this study. FIG. 2B shows post-prime endpoint titers of RBD- and N-specific IgG antibody determined by ELISA at day 21. FIG. 2C shows post-boost endpoint titers of RBD- and N-specific IgG antibody determined by ELISA at day 42, and 56. FIG. 2D shows SARS-CoV-2 surrogate virus neutralization test (sVNT) and binding/neutralizing antibody ratio at day 21 and 56. FIG. 2E shows neutralization assay using VeroE6 cells infected with authentic SARS-CoV-2. Neutralizing antibodies from O₂-Cryogel_{VAX}-treated mice were tested after prime (day 21: D21) and prime-boost (day 56: D56) immunizations. Data points show individual serum sample, and data is represented as mean ±SEM (n=5-10). *P<0.05, **P<0.01 and ***P<0.001.

FIGS. 3A-3C show O₂-Cryogel_{VAX} recruits high number of CD19+ leukocytes and stimulates the production of B cells in LNs. FIG. 3A shows photographs of cryogels and LNs at day 56 after subcutaneous injection. FIGS. 3B-3C show immune cell populations in cryogels (FIG. 3B) and LNs (FIG. 3C) as analyzed by flow cytometry. Two draining LNs and cryogels were analyzed per animal. Data points show individual LNs or cryogels, and data are represented as mean ±SEM (n=10). *P<0.05, **P<0.01 and ***P<0.001.

FIGS. 4A-4E show that immunization with O₂-Cryogel_{VAX} triggers a balanced Th1/Th2-mediated immune response against SARS-CoV-2. Endpoint titers (FIG. 4A) and (FIG. 4B) endpoint titer ratios of the different IgG subclasses after 56 days were assessed by ELISA. Th1 and Th2 cytokine levels were measured in mouse serum at day 24 (FIG. 4C) and in explanted cryogels at day 56 (FIG. 4D) by multiplex assay. FIG. 4E shows gating and frequencies of cytokine-producing CD44-positive CD4+ T cells after S and N protein-derived peptide stimulation of splenocytes isolated at day 21, as quantified by flow cytometry. Data are represented as mean ±SEM (n=5) (A-D) or values of individual spleens (n=5) (E). *P<0.05, **P<0.01 and ***P<0.001.

FIGS. 5A-5E show efficient encapsulation and sustained release of SARS-CoV-2 subunit proteins and immunomodulatory factors from cryogels. FIG. 5A shows confocal microscopy images showing co-encapsulation of Alexa Fluor 488-labeled RBD and Alexa Fluor 647-labeled N protein within the polymer walls of O₂-Cryogel_{VAX}. FIG. 5B shows cumulative release and Loading efficiency (FIG. 5C) of CpG-ODN 1826, GM-CSF and RBD in Cryogel_{VAX} and O₂-Cryogel_{VAX}. FIG. 5C shows calcium release from O₂-Cryogels, incubated at 37° C. in diH₂O, and quantified using a hardness test. FIG. 5E shows H₂O₂ release from O₂-Cryogels. Data is represented as mean ±SEM (n=3-5).

FIGS. 6A-6C show that O₂-Cryogel_{VAX} enhances the humoral immune response against SARS-CoV-2. FIGS. 6A-6B show endpoint titers of RBD- and N-specific IgM (A—day 14 and 21) and IgG (B—day 21, 42 and 56) antibody determined by ELISA. FIG. 6C shows inhibition rate as the function of serum dilution of the SARS-CoV-2 surrogate virus neutralization test at day 21 and 56. Data points show individual serum sample and data is represented as mean ±SEM (n=5). *P<0.05, **P<0.01 and ***P<0.001.

FIGS. 7A-7B show Cryogels contain limited numbers of adaptive immune cells after 56 days. FIG. 7A shows flow cytometry gating strategy and cell numbers in cryogels (FIG. 7B) at day 21 and 56 post-vaccination. Arrows indicate corresponding gels with high numbers of T cells and MHCII+B cells. Data points show individual cryogels and data is represented as mean ±SEM (n=5). *P<0.05, **P<0.01 and ***P<0.001.

FIGS. 8A-8B show that vaccination with cryogel vaccine (Cryogel_{VAX}) and O₂-Cryogel_{VAX} increases adaptive immune cell numbers in draining LNs. FIG. 8A shows flow cytometry gating strategy and cell numbers (FIG. 8B) in LNs at day 21 and 56 post-vaccination. Two draining LNs were analyzed per animal. Data points show individual LNs and data is represented as mean ±SEM. *P<0.05, **P<0.01 and ***P<0.001.

FIG. 9 shows that O₂-Cryogel_{VAX} induces IL-5-producing N-specific CD8+ T cells. Gating and frequencies of cytokine-producing CD44-positive CD8+ T cells after S and N protein-derived peptide stimulation of splenocytes isolated at day 21, as quantified by flow cytometry. Data is represented as mean ±SEM (n=5). *P<0.05, **P<0.01 and ***P<0.001.

FIG. 10 shows that O₂-cryogels enable controlled release of oxygen in their surrounding environment. Oxygen release from cryogels, APC-free (no catalase) O₂-cryogels, and O₂-cryogels (catalase containing) under normoxic conditions. Data are representative of n=4 experiments.

FIGS. 11A-11B show that immunization with O₂-Cryogel_{VAX} does not trigger an allergic response. FIG. 11A shows that mouse serum IgE titers were assessed by ELISA at day 56 across all the vaccine conditions. FIG. 11B shows that mouse serum IL-4, IL-5, IL-13 levels were measured at day 24 with a multiplex immunoassay across all the vaccine conditions. Values represent the mean ±SEM (n=5).

### DETAILED DESCRIPTION

Severe acute respiratory syndrome coronavirus 2 (SARS-CoV-2) has led to an unprecedented global health crisis, resulting in a critical need for effective vaccines that generate protective antibodies. Protein subunit vaccines represent a promising approach but often lack the immunogenicity required for strong immune stimulation. To overcome this challenge, we first demonstrate that advanced biomaterials can be leveraged to boost the effectiveness of SARS-CoV-2 protein subunit vaccines. Additionally, we report that oxygen is a powerful immunological co-adjuvant and has an ability to further potentiate vaccine potency. In preclinical studies, mice immunized with oxygen-generating cryogel (O₂-cryogel) vaccines exhibited a robust Th1 and Th2 immune response, leading to a sustained and high titer production of neutralizing antibodies against SARS-CoV-2. Even with a single immunization, O₂-cryogels achieved high antibody titers within 21 days, and both binding and neutralizing antibody levels increased after a second dose. Having a vaccine system that generates sufficient neutralizing antibodies after one dose is a preferred strategy amid vaccine shortage. Our data suggest that this platform is a promising technology to reinforce vaccine-driven immunostimulation and has great potential to be applied to other infectious diseases.

Protein subunit vaccines have been approved to protect against infectious diseases with several currently commercially available, yet they often lack the immunogenicity required to induce strong and long-lasting immunity. This is also exemplified by the recent delay of the Sanofi/GSK adjuvanted recombinant protein-based COVID-19 vaccine that demonstrated insufficient responses in older adults. Biomaterial-based delivery systems can address this challenge by enhancing vaccine immunogenicity while reducing toxicity through controlled presentation and release of antigens and immunomodulatory factors (e.g., cytokines and adjuvants). Cryogels, polymeric biomaterials with a unique interconnected macroporous network, can be used as a platform for the controlled delivery of vaccine components, as well as to recruit, host, and program immune cells in situ. A cryogel-based SARS-CoV-2 vaccine, comprising immunomodulatory factors and viral antigens, would provide an effective platform for stimulating antibody-producing B cells in the draining lymph nodes (LNs) while promoting dendritic cell (DC) activation. This strategy induces high numbers of binding and neutralizing antibodies, resulting in an effective protection against SARS-CoV-2 infection.

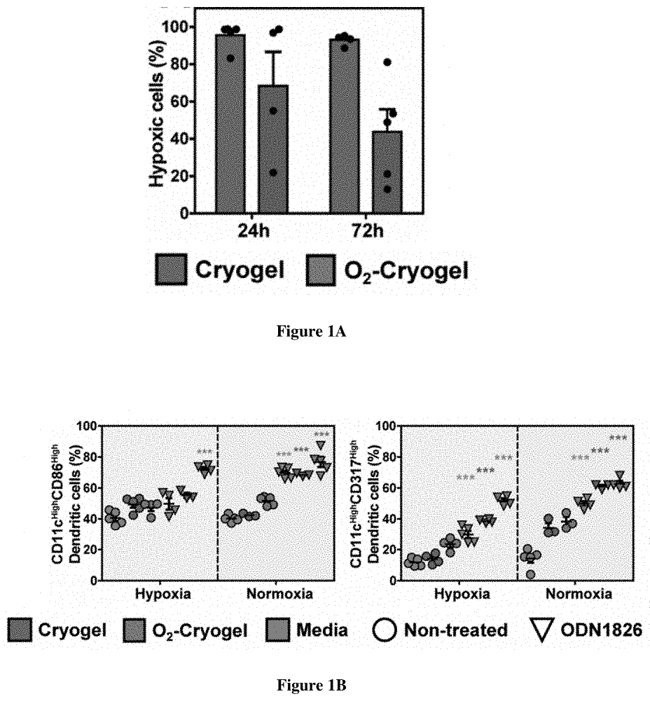

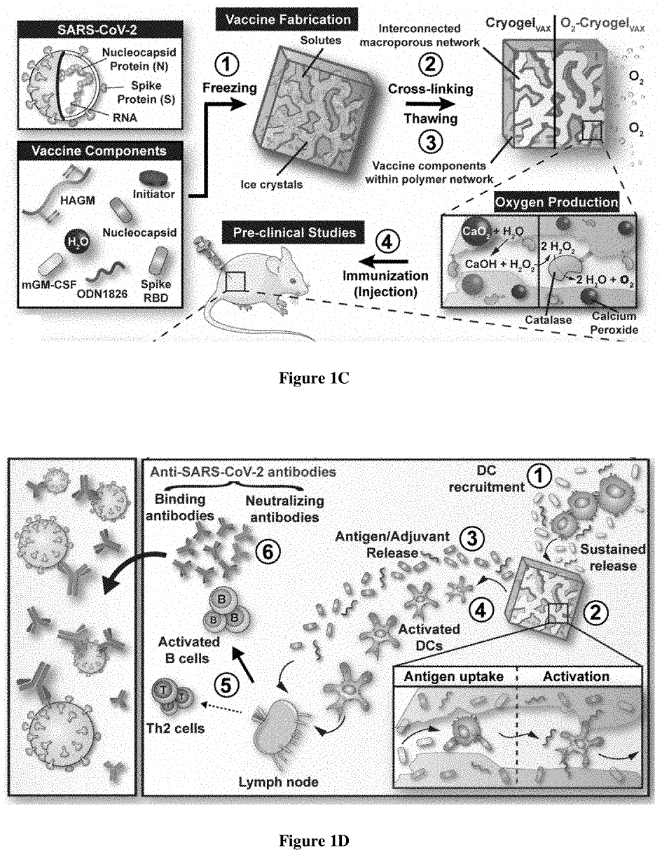

Hypoxia is a hallmark of inflamed, infected or damaged tissues due to an imbalance between oxygen supply and consumption. This low oxygen tension negatively impacts several DC functions, including survival, differentiation, migration and antigen presentation. As a result, this condition presents a major challenge when priming DCs with protein subunit vaccines. Additionally, hypoxia is also inherent to subcutaneously injected biomaterials, most likely due to a lack of local vascularization and oxygen supply (FIG. 1A). Preliminary data indicated that hypoxia alters DC activation. Therefore, we engineered oxygen-generating cryogels (O₂-cryogels) to mitigate hypoxia-driven immunosuppression. In vivo, the number of hypoxic cells was decreased within subcutaneously injected O₂-cryogels compared to standard cryogels (FIG. 1A). In vitro, O₂-cryogels restored DC activation mediated by CpG-ODN 1826, increasing the fraction of cells positive for activation markers CD86 and CD317 to levels similar to DCs stimulated under normoxic conditions (FIG. 1B). Therefore, we proposed that local oxygen supply within cryogel vaccines could potentiate SARS-CoV-2 protein subunit vaccines.

Here, to fabricate cryogel-based SARS-CoV-2 vaccines, we incorporated both the nucleocapsid protein (N), which encapsulates the virus RNA and the receptor-binding domain (RBD) of the spike protein (S) that is responsible for virus entry into cells, into hyaluronic acid-based cryogels. The cryogels also contained granulocyte macrophage colony-stimulating factor (GM-CSF), a molecule that stimulates various immune cells, including DCs, and the adjuvant CpG-ODN 1826, a ligand for TLR9 (toll-like receptor 9) that activates DCs, specifically plasmacytoid DCs (pDCs). These cryogel-based vaccines were formulated to induce a robust humoral immune response. To enhance vaccine immunogenicity, oxygen was considered as an immunological co-adjuvant that would eliminate local hypoxia at the site of vaccine administration. Thus, oxygen-producing calcium peroxide (CaO2) particles and acrylate-PEG-catalase (APC) were incorporated within the cryogel vaccine formulations before freezing. The resulting O₂-Cryogel vaccine (O₂-Cryogel_{VAX}) was designed to generate oxygen upon the reaction of CaO2 with water and to eliminate hydrogen peroxide byproducts through a catalase-mediated breakdown.

Protein antigens and adjuvants released from the vaccine are most likely draining to the inguinal lymph nodes (LNs) and direct B cell activation. Additionally, following subcutaneous immunization (FIG. 1C), cryogel-based protein subunit vaccines would induce DC-mediated humoral immunity (FIG. 1D). This is supported with findings on cryogel cancer vaccines where a sustained release of immunomodulatory factors (GM-CSF and CpG-ODN 1826) promotes DC infiltration into the macroporous network of cryogels. Within the cryogels, DCs are expected to take up protein antigens (N and RBD proteins) and to simultaneously become activated by CpG-ODN 1826 and the increased local oxygen tension. Activated, antigen-loaded DCs would then migrate to draining LNs to stimulate the activation of antigen-specific T cells and enhance activation of antibody-producing B cells. A subset of activated B cells is most likely differentiating into plasma cells with the primary role of producing large quantities of SARS-CoV-2-binding antibodies. A fraction of these antibodies would be neutralizing antibodies and exert their inhibitory activity by abrogating binding of the virus RBD to the human receptor angiotensin-converting enzyme 2 (ACE2).

Nearly every decade for the past 30 years, a novel coronavirus pandemic emerges, pushing the healthcare system to its limit. Although the current outbreak had long been predicted, SARS-CoV-2 has created the most severe crisis in recent history. The rapid development of an effective and safe vaccine against this virus is the most effective strategy to end this pandemic. Among them, protein subunit vaccines have been widely investigated against SARS-CoV-2 due to their performance and safety record, and such vaccines have already shown promising early results in phase 1/2 clinical trials. Yet, subunit vaccines still have to overcome their lack of immunogenicity. In addition, an ideal antiviral vaccine should be versatile and rapid to design, enabling rapid response to the public health emergency. To overcome these challenges, our team leveraged a cryogel-based vaccine platform to strengthen protein subunit vaccines and induce a strong and sustained anti-SARS-CoV-2 immune response. In addition, we showed that oxygen is a powerful immunological co-adjuvant that shapes and reinforces the immune response. This work demonstrated how robust and modular the cryogel-based vaccine technology is, which was successfully and quickly adapted from cancer to an infectious disease at breakneck speed (<3 months).

We found that Cryogel_{VAX} triggers both Th1 and Th2 immune responses while enhancing the efficacy of a conventional protein subunit vaccine by 100-fold (Bolus_{VAX}). This is most likely due to the ability of cryogels to control the release of immunomodulatory factors to stimulate B cells in the draining LNs while activating high numbers of immune cells within the cryogels. Following prime-boost immunization, Cryogel_{VAX} elicited a strong humoral immune response for nearly 2 months (study endpoint) and was associated with high levels of anti-RBD IgG antibodies and strong neutralizing activity to SARS-CoV-2. In addition, Cryogel_{VAX} induced CD4+ and CD8+ T cell responses, specifically directed against the N protein. The unique macroporous architecture of Cryogel_{VAX} and incorporation of a chemoattractant also promoted the recruitment of resident leukocytes and CD19+ immune cells, which likely increased B cell expansion in the LNs within 21 days. This supports the finding that Cryogel_{VAX} acts as a distant immune cell training platform that reinforces our prime-boost vaccination strategy.

Although promising, Cryogel_{VAX} has been associated with a number of limitations in this study, including a decrease in anti-SARS-CoV-2 IgG antibodies after 42 days and low concentrations of Th1 cytokines. In light of these findings, we explored the use of oxygen as an immunological co-adjuvant to potentiate vaccine efficacy. We demonstrated that supplemental oxygen not only promoted CD4+ and CD8+ T cell responses against SARS-CoV-2, but also promoted the production of Th1-biased and pro-inflammatory cytokines. Additionally, O₂-Cryogel_{VAX} remarkably boosted humoral immunity with long-lasting production of binding antibodies (5-fold higher at day 56) and high IgG1 neutralizing activity with a single injection. This suggests the induction of strong Th1 and Th2-associated immune responses. Interestingly, O₂-Cryogel_{VAX} promoted local recruitment of leukocytes, notably CD19+ cells, and enhancement of B cell expansion in the LNs. Altogether, this study clearly highlighted that oxygen can become a key vaccine co-adjuvant and play an important role in shaping the immune response, ultimately boosting vaccine efficacy.

Examining immune cell populations at earlier time points, the synergistic interaction of N and RBD proteins during immune priming, in addition to the contribution of each of the immunomodulatory factors and their optimal release kinetics, may further shed light on the vaccine mode of action. Moreover, a deeper understanding of the spatiotemporal diffusion of oxygen could be leveraged to further boost vaccine efficacy.

The study described herein unveils the magnitude of an advanced biomaterial-based technology to harness the power of protein subunit vaccines, leading to a rapid and protective anti-SARS-CoV-2 immune response. Additionally, we report the synergistic effect of vaccines engineered to provide oxygen as a powerful immunological co-adjuvant. Lastly, this platform is compatible with other strategies, such as live attenuated or inactivated pathogens and nucleic acid vaccines, and may boost the efficiency of existing vaccines or those under development. Although several vaccines have proven to be effective in phase 3 clinical trials, their stability as well as the number of required doses and the duration of their protection might not be optimal. Our study opens new possibilities to leverage vaccines, such as the one described here for COVID-19, and help quickly develop new versions as the virus could mutate and be more infectious. For instance, the recent new viral mutations of SARS-CoV-2 leading to a virus variant in Europe that is thought to spread more easily. This is a good example of the mutability of SARS-CoV-2 and its danger leading to more death, economic uncertainty, and travel restrictions. Lastly, this platform is potentially applicable in reinforcing vaccines for other infectious diseases and any conditions that may require stimulating the immune system.

In one aspect, described herein is a vaccine, comprising an oxygen-generating cryogel, wherein the oxygen-generating cryogel comprises an antigen, a chemoattractant for immune cells, a toll-like receptor 9 (TLR9) ligand, and an oxygen-producing compound. In another aspect, described herein is a method of modulating the immune system in a subject, comprising administering an effective amount of the vaccine described herein to the subject, thereby modulating the immune system in the subject. In one aspect, described herein is a method of stimulating the immune system in a subject, comprising administering an effective amount of the vaccine described herein to the subject, thereby modulating the immune system in the subject.

In one aspect, described herein is a method comprising administering to a subject the vaccine described herein, wherein the vaccine is administered to the subject in an effective amount to induce an immune response in the subject. In one aspect, described herein is a method of preventing an infection in a subject comprising administering an effective amount of the vaccine described herein to the subject.

Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

B cells, also known as B lymphocytes, are a type of white blood cell of the lymphocyte subtype. They function in the humoral immunity component of the adaptive immune system. B cells mediate the production of antigen-specific immunoglobulin (Ig) directed against invasive pathogens (typically known as antibodies). The antibodies are inserted into the plasma membrane where they serve as a part of B-cell receptors. When a naïve or memory B cell is activated by an antigen, it proliferates and differentiates into an antibody-secreting effector cell, known as a plasmablast or plasma cell. Additionally, B cells present antigens (they are also classified as professional antigen-presenting cells (APCs)) and secrete cytokines.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a viral infection, is well understood in the art, and includes administration of a composition which reduces the frequency or severity of, or delays the onset of, symptoms of a medical condition in a subject relative to a subject which does not receive the composition. Thus, prevention of coronavirus infection includes, for example, reducing the incidence of coronavirus infection in a population of patients receiving a prophylactic treatment relative to an untreated control population, and/or delaying the incidence and/or reducing the severity of coronavirus infection in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

As used herein, the term “biocompatible” refers to materials that are, with any metabolites or degradation products thereof, generally non-toxic and cause no significant adverse effects to living cells and tissues.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skill in the art to result in chemically stable compounds which can be readily synthesized, e.g., by techniques known in the art, as well as those methods set forth below, from readily available starting materials. If a substituent is itself substituted with more than one group, it is understood that these multiple groups may be on the same carbon or on different carbons, so long as the structure is stable.

The phrase “pharmaceutically acceptable carrier” as used herein means a pharmaceutically acceptable material, composition, or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

As used herein, the term “coronavirus” refers to a virus belonging to the subfamily Orthocoronavirinae, in the family Coronaviridae, order Nidovirales, and realm Riboviria. A coronavirus is an enveloped virus with a positive-sense single-stranded RNA genome and a nucleocapsid of helical symmetry. Coronaviruses include, for example, the following human viruses: human coronavirus 229E, human coronavirus OC43, SARS-CoV, HCoV, HKU1, MERS-CoV, and SARS-CoV-2. In some embodiments, coronavirus is SARS-CoV-2.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

Cryogel

PCT application publication No. WO 2020/033713 describes methods of making oxygen-generating cryogels; the published patent application is hereby incorporated by reference in its entirety.

Any biocompatible polymers or monomers undergoing cryopolymerization could be used to make cryogels, including but not limited to naturally derived polymers (alginate, hyaluronic acid, heparin, gelatin, carob gum, collagen, peptides, proteins, etc) and synthetic polymers (poly(ethylene glycol) (PEG), PEGylated glutaminase (PEG-PGA), PEG-poly(L-lactide; PLA), poly(2-hydroxyethyl methacrylate) (pHEMA), PAAm, poly(N-isopropylacrylamide) (PNIPAAm), etc.). For example, the polymers may be a combination of degradable and non-degradable synthetic polymers and natural polymers (polysaccharides, peptides, proteins, DNA). Biocompatible synthetic polymers include Polyethylene glycol (PEG), Polyvinyl alcohol (PVA), Poly(2-hydroxyethyl methacrylate) (PHEMA), Poly(N-isopropylacrylamide) (PNIPAAm), Poly(acrylic acid) (PAAc), Polyesters (e.g. Polylactide, Polyglycolide, Polycaprolactone), and Polyanhydrides. Naturally occurring polymers include Carbohydrates (e.g. Starch, Cellulose, Dextrose, Alginate, Hyaluronic Acid, Heparin, Dextran, Gellan Gum, etc), Proteins (e.g. Gelatin, Albumin, Collagen), Peptides, and DNA. All compositions are purified prior to fabrication of the hydrogels.

In addition to the free radical polymerization process to crosslink the polymers and make chemically crosslinked injectable cryogels (polymerization time is about 17 h), cryogels are optionally polymerized using other processes. Injectable cryogels can be classified under two main groups according to the nature of their crosslinking mechanism, namely chemically and physically crosslinked gels. Covalent crosslinking processes include radical polymerization (vinyl-vinyl coupling), Michael-type addition reaction (vinyl-thiol crosslinking), Condensation (carboxylic acid-alcohol and carboxylic acid-amine crosslinking), Oxidation (thiol-thiol crosslinking), Click chemistry (1,3-dipolar cycloaddition of organic azides and alkynes), Diels-Alder reaction (cycloaddition of dienes and dienophiles), Oxime, Imine and Hydrazone chemistries. Non-covalent crosslinking include Ionic crosslinking (e.g. calcium-crosslinked alginate), Self-assembly (phase transition in response to external stimuli, such as Temperature, pH, ion concentration, hydrophobic interactions, light, metabolite, and electric current).

The O₂-cryogel vaccine technology is compatible, and can be combined with, other vaccine technologies (e.g., mRNA-based vaccines, inactivated vaccines, live-attenuated vaccines, saponin-based vaccines, non-replicating viral vector vaccines, recombinant vaccines, subunit vaccine) for a synergistic effect, i.e., boost vaccine efficacy and induce faster immunoprotection.

Cryogel vaccines can be used in different sizes (micrometer: 200-1000um to millimeter: 1-100mm scale), volumes (100 um³ to 500 mm³), and shapes (spherical, cuboidal, ellipsoidal, cylindrical.

Cryogel vaccines have shape memory properties following deformation by compression or dehydration.

Cryogel vaccines can be degraded in vivo via various mechanisms including hydrolysis, oxidation, enzymatic.

Dendritic Cells

Dendritic cells (DCs) are immune cells within the mammalian immune system and are derived from hematopoietic bone marrow progenitor cells. More specifically, dendritic cells can be categorized into lymphoid (or plasmacytoid) dendritic cell (pDC) and myeloid dendritic cell (mDC) subdivisions having arisen from a lymphoid (or plasmacytoid) or myeloid precursor cell, respectively. From the progenitor cell, regardless of the progenitor cell type, an immature dendritic cell is born. Immature dendritic cells are characterized by high endocytic activity and low T-cell activation potential. Thus, immature dendritic cells constitutively sample their immediate surrounding environment for pathogens. Exemplary pathogens include, but are not limited to, a virus or a bacteria. Sampling is accomplished by pattern recognition receptors (PRRs) such as the toll-like receptors (TLRs). Dendritic cells activate and mature once a pathogen is recognized by a pattern recognition receptor, such as a toll-like receptor.

Mature dendritic cells not only phagocytose pathogens and break them down, but also, degrade their proteins, and present pieces of these proteins, also referred to as antigens, on their cell surfaces using MHC (Major Histocompatibility Complex) molecules (Classes I, II, and III). Mature dendritic cells also upregulate cell-surface receptors that serve as co-receptors for T-cell activation. Exemplary co-receptors include, but are not limited to, CD80, CD86, and CD40. Simultaneously, mature dendritic cells upregulate chemotactic receptors, such as CCR7, that allows the cell to migrate through the blood stream or the lymphatic system to the spleen or lymph node, respectively.

Dendritic cells are present in external tissues that are in contact with the external environment such as the skin (dendritic cells residing in skin are also referred to as Langerhans cells). Alternatively, dendritic cells are present in internal tissues that are in contact with the external environment such as linings of the nose, lungs, stomach, and intestines. Finally, immature dendritic cells reside in the blood stream. Once activated, dendritic cells from all off these tissues migrate to lymphoid tissues where they present antigens and interact with T cells and B cells to initiate an immune response. One signaling system of particular importance for the present invention involves the chemokine receptor CCR7 expressed on the surface of dendritic cells and the chemokine receptor ligand CCL19 secreted by lymph node structures to attract migrating mature dendritic cells toward high concentrations of immune cells. Exemplary immune cells activated by contact with mature dendritic cells include, but are not limited to, helper T cells, killer T cells, and B cells. Although multiple cell types within the immune system present antigens, including macrophages and B lymphocytes, dendritic cells are the most potent activators of all antigen-presenting cells.

Adjuvant

In immunology, an adjuvant is a substance that increases and/or modulates the immune response to a vaccine. An immunologic adjuvant is defined as any substance that acts to accelerate, prolong, or enhance antigen-specific immune responses when used in combination with specific vaccine antigens.

Adjuvants are used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called pathogen-associated molecular patterns, which include liposomes, lipopolysaccharide, molecular cages for antigens, components of bacterial cell walls, and endocytosed nucleic acids such as double-stranded RNA, double-stranded RNA, single-stranded DNA, and unmethylated CpG dinucleotide-containing DNA. The presence of an adjuvant in conjunction with the vaccine can greatly increase the innate immune response to the antigen by augmenting the activities of dendritic cells, lymphocytes, and macrophages by mimicking a natural infection.

There are many known adjuvants in widespread use, including aluminum salts, oils and virosomes. In some embodiments, the adjuvant is an inorganic compound, such as potassium alum, aluminum hydroxide, aluminum phosphate, calcium phosphate hydroxide. In some embodiments, the adjuvant is an oil, such as paraffin oil, and propolis. In some embodiments, the adjuvant is a bacterial product, such as killed bacteria of the species Bordetella pertussis, Mycobacterium bovis, and toxoids. In some embodiments, the adjuvant is a plant saponin from Quillaja, soybean, or Polygala senega. In some embodiments, the adjuvant is a cytokines, such as IL-1, IL-2, and IL-12. In some embodiments, the adjuvant is Freund's complete adjuvant, or Freund's incomplete adjuvant. In some embodiments, the adjuvant is squalene.

Various immunologic adjuvants could be used, including but not limited to lipopeptides, glycolipids, proteolipids, viruses (e.g., double-stranded RNA, poly I:C, single-stranded RNA), lipopolysaccharides (e.g., monophosphoryl-lipid A), proteins (e.g., Heat shock proteins, Profilin), mycoplasma (e.g., multiple diacyl lipopeptides), bacteria (e.g., bacterial flagellin, lipoteichoic acid, multiple triacyl lipopeptides, bacterial ribosomal RNA sequence “CGGAAAGACC”), fungi (e.g., zymosan (Beta-glucan)), peptides, polysaccharides (e.g., hyaluronic acid fragments, heparan sulfate fragments), small synthetic molecules (e.g., imidazoquinoline, resiquimod, bropirimine, opioid drugs), toxoplasma gondii (e.g., profilin), oils (e.g., paraffin oil, propolis, adjuvant 65), inorganic molecules (e.g., aluminum salts).

Nucleic acid adjuvants such as unmethylated CpG Oligodeoxynucleotide DNA can be condensed with transfection agents (viral and nonviral) such as stable cationic molecules/polymers. One example is polyethylenimine (PEI). PEI condenses CpG into positively charged particles that bind to anionic cell surfaces. Nonviral methods include chemical-based transfection agents (e.g., calcium phosphate, cationic polymer, liposomes, dendrimers), particle-based transfection agents (e.g., nanoparticles, nanowires, nanofibers). Viral methods include viruses and adenovirus vectors.

All toll-like receptors (TLRs) could be used including TLR1, TLR2, TLR3, TLR4, TLR5, TLR6, TLR7, TLR8, TLR9, TLR10, TLR11, TLR12, and TLR13.

In some embodiments, the adjuvant is a toll-like receptor (TLR) ligand. TLRs are a class of single transmembrane domain, non-catalytic, receptors that recognize structurally conserved molecules referred to as pathogen-associated molecular patterns (PAMPs). PAMPs are present on microbes and are distinguishable from host molecules. TLRs are present in all vertebrates. Thirteen TLRs (referred to as TLRs 1-13, consecutively) have been identified in humans and mice. Humans comprise TLRs 1-10.

TLRs and interleukin-1 (IL-1) receptors comprise a receptor superfamily the members of which all share a TIR domain (Toll-IL-1 receptor). TIR domains exist in three varieties with three distinct functions. TIR domains of subgroup 1 are present in receptors for interleukins produced by macrophages, monocytes, and dendritic cells. TIR domains of subgroup 2 are present in classical TLRs which bind directly or indirectly to molecules of microbial origin. TIR domains of subgroup 3 are present in cytosolic adaptor proteins that mediate signaling between proteins comprising TIR domains of subgroups 1 and 2.

TLR ligands comprise molecules that are constantly associated with and highly specific for a threat to the host's survival such as a pathogen or cellular stress. TLR ligands are highly specific for pathogens and not the host. Exemplary pathogenic molecules include, but are not limited to, lipopolysaccharides (LPS), lipoproteins, lipoarabinomannan, flagellin, double-stranded RNA, and unmethylated CpG islands of DNA.

In one preferred embodiment of the present invention, the Toll-Like receptor 9 (TLR9) is activated by specific unmethylated CpG-containing sequences in bacterial DNA or synthetic oligonucleotides (ODNs) found in the endosomal compartment of dendritic cells. Methylation status of the CpG site is a crucial distinction between bacterial and mammalian DNA, as well as between normal and cancerous tissue. Unmethylated ODNs including one or more CpG motifs mimic the effects of bacterial DNA. Alternatively, or in addition, unmethylated ODNs including one or more CpG motifs occur within oncogenes present within malignant tumor cells.

One or more sequences of the TLR-9 receptor recognizes one or more CpG-ODN sequences of the present invention. Human TLR-9, isoform A, is encoded by the mRNA sequence (NCBI Accession No. NM_017442, incorporated herein by reference). Human TLR-9, isoform A, is encoded by the amino acid sequence (NCBI Accession No. NP 059138, incorporated herein by reference). Human TLR3 is encoded by the mRNA sequence (GenBank Accession No. NM_003265.2 (GI:19718735), incorporated herein by reference). Human TLR3 is encoded by the amino acid sequence (GenBank Accession No. ABC86910.1 (GI:86161330), incorporated herein by reference).

Granulocyte Macrophage Colony Stimulating Factor (GM-CSF)

Granulocyte-macrophage colony-stimulating factor (GM-CSF) is a protein secreted by macrophages, T cells, mast cells, endothelial cells and fibroblasts. Specifically, GM-CSF is a cytokine that functions as a white blood cell growth factor. GM-CSF stimulates stem cells to produce granulocytes and monocytes. Monocytes exit the blood stream, migrate into tissue, and subsequently mature into macrophages.

Cryogels described herein comprise and release GM-CSF polypeptides to attract host DCs to the device. Contemplated GM-CSF polypeptides are isolated from endogenous sources or synthesized in vivo or in vitro. Endogenous GM-CSF polypeptides are isolated from healthy human tissue. Synthetic GM-CSF polypeptides are synthesized in vivo following transfection or transformation of template DNA into a host organism or cell, e.g. a mammal or cultured human cell line. Alternatively, synthetic GM-CSF polypeptides are synthesized in vitro by polymerase chain reaction (PCR) or other art-recognized methods Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference).

GM-CSF polypeptides are modified to increase protein stability in vivo. Alternatively, GM-CSF polypeptides are engineered to be more or less immunogenic. Endogenous mature human GM-CSF polypeptides are glycosylated, reportedly, at amino acid residues 23 (leucine), 27 (asparagine), and 39 (glutamic acid) (see U.S. Pat. No. 5,073,627). GM-CSF polypeptides of the present invention are modified at one or more of these amino acid residues with respect to glycosylation state.

GM-CSF polypeptides are recombinant. Alternatively GM-CSF polypeptides are humanized derivatives of mammalian GM-CSF polypeptides. Exemplary mammalian species from which GM-CSF polypeptides are derived include, but are not limited to, mouse, rat, hamster, guinea pig, ferret, cat, dog, monkey, or primate. In a preferred embodiment, GM-CSF is a recombinant human protein (PeproTech, Catalog #300-03). Alternatively, GM-CSF is a recombinant murine (mouse) protein (PeproTech, Catalog #315-03). Finally, GM-CSF is a humanized derivative of a recombinant mouse protein.

Cytosine-Guanosine (CpG) Oligonucleotide (CpG-ODN) Sequences

CpG sites are regions of deoxyribonucleic acid (DNA) where a cysteine nucleotide occurs next to a guanine nucleotide in the linear sequence of bases along its length (the “p” represents the phosphate linkage between them and distinguishes them from a cytosine-guanine complementary base pairing). CpG sites play a pivotal role in DNA methylation, which is one of several endogenous mechanisms cells use to silence gene expression. Methylation of CpG sites within promoter elements can lead to gene silencing. In the case of cancer, it is known that tumor suppressor genes are often silences while oncogenes, or cancer-inducing genes, are expressed. Importantly, CpG sites in the promoter regions of tumor suppressor genes (which prevent cancer formation) have been shown to be methylated while CpG sites in the promoter regions of oncogenes are hypomethylated or unmethylated in certain cancers. The TLR-9 receptor binds unmethylated CpG sites in DNA.

The present invention comprises CpG dinucleotides and oligonucleotides. Contemplated CpG oligonucleotides are isolated from endogenous sources or synthesized in vivo or in vitro. Exemplary sources of endogenous CpG oligonucleotides include, but are not limited to, microorganisms, bacteria, fungi, protozoa, viruses, molds, or parasites. Alternatively, endogenous CpG oligonucleotides are isolated from mammalian benign or malignant neoplastic tumors. Synthetic CpG oligonucleotides are synthesized in vivo following transfection or transformation of template DNA into a host organism. Alternatively, Synthetic CpG oligonucleotides are synthesized in vitro by polymerase chain reaction (PCR) or other art-recognized methods (Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference).

CpG oligonucleotides are presented for cellular uptake by dendritic cells. In one embodiment, naked CpG oligonucleotides are used. The term “naked” is used to describe an isolated endogenous or synthetic polynucleotide (or oligonucleotide) that is free of additional substituents. In another embodiment, CpG oligonucleotides are bound to one or more compounds to increase the efficiency of cellular uptake. Alternatively, or in addition, CpG oligonucleotides are bound to one or more compounds to increase the stability of the oligonucleotide within the scaffold and/or dendritic cell.

CpG oligonucleotides are condensed prior to cellular uptake. The unmethylated CpG Oligodeoxynucleotide DNA can be condensed with transfection agents (viral and nonviral) such as stable cationic molecules/polymers. One example is polyethylenimine (PEI). PEI condenses CpG into positively charged particles that bind to anionic cell surfaces. PEI is a cationic polymer that increases the efficiency of cellular uptake into dendritic cells. Nonviral methods include chemical-based transfection agents (e.g., calcium phosphate, cationic polymer, liposomes, dendrimers), particle-based transfection agents (e.g., nanoparticles, nanowires, nanofibers). Viral methods include viruses and adenovirus vectors.

Three major classes of stimulatory CpG ODNs have been identified based on structural characteristics and activity on human peripheral blood mononuclear cells (PBMCs), in particular B cells and plasmacytoid dendritic cells (pDCs). These three classes are Class A (Type D), Class B (Type K) and Class C.

CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production.

CpG-B ODNs contain a full PS backbone with one or more CpG dinucleotides. They strongly activate B cells and TLR9-dependent NF-κB signaling but weakly stimulate IFN-α secretion.

CpG-C ODNs combine features of both classes A and B. They contain a complete PS backbone and a CpG-containing palindromic motif. C-Class CpG ODNs induce strong IFN-α production from pDC as well as B cell stimulation

CpG oligonucleotides of the present invention can be divided into multiple classes. For example, exemplary CpG-ODNs encompassed by compositions, methods and devices of the present invention are stimulatory, neutral, or suppressive. The term “stimulatory” used herein is meant to describe a class of CpG-ODN sequences that activate TLR9. The term “neutral” used herein is meant to describe a class of CpG-ODN sequences that do not activate TLR9. The term “suppressive” used herein is meant to describe a class of CpG-ODN sequences that inhibit TLR9. The term “activate TLR9” describes a process by which TLR9 initiates intracellular signaling.

Simulatory CpG-ODNs can further be divided into three types A, B and C, which differ in their immune-stimulatory activities. Type A stimulatory CpG ODNs are characterized by a phosphodiester central CpG-containing palindromic motif and a phosphorothioate 3′ poly-G string. Following activation of TLR9, these CpG ODNs induce high IFN-α production from plasmacytoid dendritic cells (pDC). Type A CpG ODNs weakly stimulate TLR9-dependent NF-κB signaling.

Type B stimulatory CpG ODNs contain a full phosphorothioate backbone with one or more CpG dinucleotides. Following TLR9 activation, these CpG-ODNs strongly activate B cells. In contrast to Type A Cpg-ODNs, Type B CpG-ODNS weakly stimulate IFN-α secretion.

Type C stimulatory CpG ODNs comprise features of Types A and B. Type C CpG-ODNs contain a complete phosphorothioate backbone and a CpG containing palindromic motif. Similar to Type A CpG ODNs, Type C CpG ODNs induce strong IFN-α production from pDC. Similar to Type B CpG ODNs, Type C CpG ODNs induce strong B cell stimulation.

Exemplary stimulatory CpG ODNs comprise, but are not limited to, ODN 1585, ODN 1668, ODN 1826, ODN 2006, ODN 2006-G5, ODN 2216, ODN 2336, ODN 2395, ODN M362 (all InvivoGen). The present invention also encompasses any humanized version of the preceding CpG ODNs. In one preferred embodiment, compositions, methods, and devices of the present invention comprise ODN 1826 (the sequence of which from 5′ to 3′ is tccatgacgttcctgacgtt, wherein CpG elements are bolded, SEQ ID NO: 10).

Neutral, or control, CpG ODNs that do not stimulate TLR9 are encompassed by the present invention. These ODNs comprise the same sequence as their stimulatory counterparts but contain GpC dinucleotides in place of CpG dinucleotides.

Exemplary neutral, or control, CpG ODNs encompassed by the present invention comprise, but are not limited to, ODN 1585 control, ODN 1668 control, ODN 1826 control, ODN 2006 control, ODN 2216 control, ODN 2336 control, ODN 2395 control, ODN M362 control (all InvivoGen). The present invention also encompasses any humanized version of the preceding CpG ODNs.

Suppressive CpG ODNs that inhibit TLR9 are encompassed by the present invention. Exemplary potent inhibitory sequences are (TTAGGG)4 (ODN TTAGGG, InvivoGen), found in mammalian telomeres and ODN 2088 (InvivoGen), derived from a murine stimulatory CpG ODN by replacement of 3 bases. Suppressive ODNs disrupt the colocalization of CpG ODNs with TLR9 in endosomal vesicles without affecting cellular binding and uptake. Suppressive CpG ODNs encompassed by the present invention are used to fine-tune, attenuate, reverse, or oppose the action of a stimulatory CpG-ODN. Alternatively, or in addition, compositions, methods, or devices of the present invention comprising suppressive CpG ODNs are used to treat autoimmune conditions or prevent immune responses following transplant procedures.

Chemoattractants

The cryogel of the present invention can comprise a chemoattractant for cells. The term “chemoattractant,” as used herein, refers to any agent that attracts a motile cell, such as immune cells. To enable sustained release from the scaffold, the chemoattractant can, in some embodiments, be coupled to nanoparticles, e.g., gold nanoparticles.

In certain embodiments, the chemoattractant for immune cells is a growth factor or cytokine. In some embodiments, the chemoattractant is a chemokine. Exemplary chemokines include, but are not limited to, CC chemokines, CXC chemokines, C chemokines, CX3C chemokines. Exemplary cytokines include, but are not limited to, interleukin, lymphokines, monokines, interferons, and colony stimulating factors. All known growth factors are encompassed by the compositions, methods, and devices of the present invention. Exemplary growth factors include, but are not limited to, transforming growth factor beta (TGF-b), granulocyte-colony stimulating factor (G-CSF), granulocyte-macrophage colony stimulating factor (GM-CSF), nerve growth factor (NGF), neurotrophins, Platelet-derived growth factor (PDGF), erythropoietin (EPO), thrombopoietin (TPO), myostatin (GDF-8), growth differentiation factor-9 (GDF9), acidic fibroblast growth factor (aFGF or FGF-1), basic fibroblast growth factor (bFGF or FGF-2), epidermal growth factor (EGF), hepatocyte growth factor (HGF). In some embodiments, the cryogel includes a chemoattractant for immune cells. In some embodiments, the cryogel comprises a compound that attracts an immune cell to or into the cryogel, wherein the immune cell comprises a macrophage, T-cell, B-cell, natural killer (NK) cell, or dendritic cell. Non-limiting examples of compounds useful for attracting an immune cell to or into the cryogel comprises granulocyte-macrophage colony stimulating factor (GM-CSF), an FMS-like tyrosine kinase 3 ligand (Flt3L), chemokine (C-C motif) ligand 19 (CCL-19), chemokine (C-C motif) ligand 20 (CCL20), chemokine (C-C motif) ligand 21 (CCL-21), a N-formyl peptide, fractalkine, monocyte chemotactic protein-1, and macrophage inflammatory protein-3 (MIP-3a). The present invention encompasses cytokines as well as growth factors for stimulating dendritic cell activation. Exemplary cytokines include, but are not limited to, IL-1, IL-2, IL-3, IL-4, IL-5, IL-6, IL-8, IL-10, IL-12 1L-15, IL-17, IL-18, IL-23, TNFα, IFNγ, and IFNα.

In certain embodiments, the chemoattractant for immune cells is Granulocyte-macrophage colony-stimulating factor (GM-CSF). Granulocyte-macrophage colony- stimulating factor (GM-CSF) is a protein secreted by macrophages, T cells, mast cells, endothelial cells and fibroblasts. Specifically, GM-CSF is a cytokine that functions as a white blood cell growth factor. GM-CSF stimulates stem cells to produce granulocytes and monocytes. Monocytes exit the blood stream, migrate into tissue, and subsequently mature into macrophages.

In some embodiments, the cryogel can comprise and release GM-CSF polypeptides to attract host DCs to the device. Contemplated GM-CSF polypeptides are isolated from endogenous sources or synthesized in vivo or in vitro. Endogenous GM-CSF polypeptides may be isolated from healthy human tissue. Synthetic GM-CSF polypeptides are synthesized in vivo following transfection or transformation of template DNA into a host organism or cell, e.g., a mammalian or human cell line. Alternatively, synthetic GM-CSF polypeptides are synthesized in vitro by polymerase chain reaction (PCR) or other art-recognized methods Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference).

In certain embodiments, GM-CSF polypeptides may be recombinant. In some embodiments, GM-CSF polypeptides are humanized derivatives of mammalian GM-CSF polypeptides. Exemplary mammalian species from which GM-CSF polypeptides are derived include, but are not limited to, mouse, rat, hamster, guinea pig, ferret, cat, dog, monkey, or primate. In some embodiments, GM-CSF is a recombinant human protein (PeproTech, Catalog # 300-03). In some embodiments, GM-CSF is a recombinant murine (mouse) protein (PeproTech, Catalog #315-03). In some embodiments, GM-CSF is a humanized derivative of a recombinant mouse protein.

In certain embodiments, GM-CSF polypeptides may be modified to increase protein stability in vivo. In certain embodiments, GM-CSF polypeptides may be engineered to be more or less immunogenic. Endogenous mature human GM-CSF polypeptides are glycosylated, reportedly, at amino acid residues 23 (leucine), 27 (asparagine), and 39 (glutamic acid) (see U.S. Pat. No. 5,073,627). In certain embodiments, GM-CSF polypeptides of the present invention may be modified at one or more of these amino acid residues with respect to glycosylation state.

The chemoattractant for immune cells may recruit immune cells to the scaffolds of the present invention. Immune cells include cells of the immune system that are involved in immune response. Exemplary immune cells includes, but not limited to, T cells, B cells, leucocytes, lymphocytes, antigen presenting cells, dendritic cells, neutrophils, eosinophils, basophils, monocytes, macrophages, histiocytes, mast cells, and microglia.

In certain embodiments, the chemoattractant for immune cells recruits dendritic cells (DCs) to the scaffold of the present invention. Dendritic cells (DCs) are immune cells within the mammalian immune system and are derived from hematopoietic bone marrow progenitor cells. More specifically, dendritic cells can be categorized into lymphoid (or plasmacytoid) dendritic cell (pDC) and myeloid dendritic cell (mDC) subdivisions having arisen from a lymphoid (or plasmacytoid) or myeloid precursor cell, respectively. From the progenitor cell, regardless of the progenitor cell type, an immature dendritic cell is born. Immature dendritic cells are characterized by high endocytic activity and low T-cell activation potential. Thus, immature dendritic cells constitutively sample their immediate surrounding environment for pathogens. Exemplary pathogens include, but are not limited to, a virus or a bacteria.

Sampling is accomplished by pattern recognition receptors (PRRs) such as the toll-like receptors (TLRs). Dendritic cells activate and mature once a pathogen is recognized by a pattern recognition receptor, such as a toll-like receptor.

Mature dendritic cells not only phagocytose pathogens and break them down, but also, degrade their proteins, and present pieces of these proteins, also referred to as antigens, on their cell surfaces using MHC (Major Histocompatibility Complex) molecules (Classes I, II, and III). Mature dendritic cells also upregulate cell-surface receptors that serve as co- receptors for T-cell activation. Exemplary co-receptors include, but are not limited to, CD80, CD86, and CD40. Simultaneously, mature dendritic cells upregulate chemotactic receptors, such as CCR7, that allows the cell to migrate through the blood stream or the lymphatic system to the spleen or lymph node, respectively.

Dendritic cells are present in external tissues that are in contact with the external environment such as the skin (dendritic cells residing in skin are also referred to as Langerhans cells). Alternatively, dendritic cells are present in internal tissues that are in contact with the external environment such as linings of the nose, lungs, stomach, and intestines. Finally, immature dendritic cells reside in the blood stream. Once activated, dendritic cells from all off these tissues migrate to lymphoid tissues where they present antigens and interact with T cells and B cells to initiate an immune response. One signaling system of particular importance for the present invention involves the chemokine receptor CCR7 expressed on the surface of dendritic cells and the chemokine receptor ligand CCL19 secreted by lymph node structures to attract migrating mature dendritic cells toward high concentrations of immune cells. Exemplary immune cells activated by contact with mature dendritic cells include, but are not limited to, helper T cells, killer T cells, and B cells.

Although multiple cell types within the immune system present antigens, including macrophages and B lymphocytes, dendritic cells are the most potent activators of all antigen-presenting cells.

Dendritic cells earned their name from the characteristic cell shape comprising multiple dendrites extending from the cell body. The functional benefit of this cell shape is a significantly increased cell surface and contact area to the surroundings compared to the cell volume. Immature dendritic cells sometimes lack the characteristic dendrite formations and are referred to as veiled cells. Veiled cells possess large cytoplasmic veils rather than dendrites.

### EXAMPLES

The invention now being generally described, it will be more readily understood by reference to the following examples, which are included merely for purposes of illustration of certain aspects and embodiments of the present invention, and are not intended to limit the invention.

Example 1: Methods

Cryogel fabrication

Cryogels were fabricated as previously described by redox-induced free radical cryopolymerization of hyaluronic acid glycidyl methacrylate (HAGM-4% wt/vol) at subzero temperature (−20° C.). Briefly, the polymer solution was precooled at 4° C. prior to adding tetramethylethylenediamine (TEMED-0.42% wt/vol, Sigma-Aldrich) and ammonium persulfate (APS-0.84% wt/vol, Sigma Aldrich). Then, the mixture was transferred into Teflon® molds (4×4×1 mm, cubiform with 2 square-shaped sides, 16 μL), placed in a freezer at −20° C., and allowed to cryopolymerize for 16 h. Finally, the newly formed cryogels were thawed at room temperature (RT) to remove ice crystals and washed with Dulbecco's Phosphate Buffered Saline (DPBS, Gibco). For O₂-cryogel fabrication, acrylate-PEG-catalase (APC-1% wt/vol, Sigma-Aldrich) and calcium peroxide (CaO₂-1% wt/vol) were mixed with the cryogel polymer solution before the addition of TEMED and APS as previously reported.

SARS-CoV-2 vaccine fabrication

Protein subunit-based vaccines were fabricated by formulating (1) purified recombinant SARS-CoV-2 Spike (ATM) his-tagged protein (RBD, 10 YP_009724390.1-Arg319-Phe541, Creative Biomart nCoVS-125V), (2) purified recombinant 2019-nCoV Nucleocapsid protein (N, YP_009724397.2, Creative Biomart N-127V), purified recombinant mouse granulocyte macrophage colony stimulating factor (mGM-CSF, GenScript), and synthetic immunostimulatory oligonucleotide containing unmethylated CpG dinucleotides (CpG ODN 1826, 5′-tccatgacgttcctgacgtt-3′, VacciGrade, InvivoGen) in DPBS. For Bolus_{VAX}, 10 μg RBD, 10 μg N, 1.5 μg GM-CSF, and 50 μg CpG ODN 1826 were formulated in 100 μL of DPBS. For Cryogel_{VAX} and O₂-Cryogel_{VAX}, 10 μg RBD, 10 μg N, 1.5 μg GM-CSF, and 50 μg CpG ODN 1826 (per gel) were incorporated within the polymer solution prior to cryogelation. After thawing, each cryogel-based vaccine was resuspended in 100 μL of DPBS. For Freund_{VAX} (positive control), 25 μg RBD, 25 μg N, and 3 μg mGM-CSF were formulated in 50 μL DPBS and mixed at a 1:1 ratio with complete Freund's adjuvant (CFA-Prime) or incomplete Freund's adjuvant (IFA-Boost). Sham vaccine formulation containing only 100 μL DPBS was used as a negative control.

Mouse model and study design

Animal experiments were carried out in compliance with the National Institutes of Health (NIH) guidelines and approved by the Division of Laboratory Animal Medicine and Northeastern University Institutional Animal Care and Use Committee (protocol number 20-0629R). Vaccination studies were performed on 6-8-week-old female BALB/c (Charles River). Freund_{VAX} was inoculated intraperitoneally (IP) (1 injection/mouse). Sham, Bolus_{VAX}, Cryogel_{VAX}, and O₂-Cryogel_{VAX} were injected subcutaneously (s.c.) in both flanks (total of 2 injections/mouse). Boost injections were performed 21 days after priming at the same location. Blood samples were collected every seven days from day 14 onwards and three days post-boost (day 24). Cryogel-based vaccines, LNs, and spleens were harvested at day 21 (prime) and day 56 (prime+boost) and then dissociated as previously described. Hypoxia studies were performed on 6-8-week-old female C57BL/6 mice (Charles River). Cryogels and O₂-cryogels were suspended in 100 μL DPBS and injected s.c. into mouse flanks. After 23 h or 71 h, mice were injected IP with 200 μL of hypoxyprobe in DPBS (dosage: 60 mg/kg, hypoxyprobe), and 1 h after administration, the Cryogels and O₂-cryogels were harvested, dissociated, and stained with FITC-MAb1 following manufacturer's recommendation. The number of hypoxic cells was then analyzed via flow cytometry using an Attune N×T flow cytometer (ThermoFisher).

BMDC generation for DC activation studies

Dendritic cells (DC) activation studies were performed using bone marrow-derived dendritic cells (BMDCs) generated from 6-8-week-old female C57BL/6 mice (Charles River) as previously described. Briefly, femurs of mice were explanted, disinfected in 70% ethanol for 5 min, washed in DPBS, and then bone ends were removed, and the marrow flushed with DPBS (2 mL, 27G needle). Next, cells were mechanically dissociated by pipetting, centrifuged (5 min, 300 g), and resuspended (10⁶ cells/mL) in Roswell Park Memorial Institute Medium (RPMI 1640, Gibco) supplemented with 10% heat-inactivated fetal bovine serum (FBS, Sigma Aldrich), 100 U/mL penicillin (Gibco), 100 μg/mL streptomycin (Gibco), 2×10⁻³ M L-glutamine (Gibco), and 50×10⁻⁶ M 2-mercaptoethanol (Gibco). At day 0, bone marrow-derived cells were seeded in non-treated p6 well plates (2×10⁶ cells per well) in 5 mL of complete RPMI medium supplemented with 20 ng/mL mGM-CSF. At day 3, another 5 mL of RPMI medium containing 20 ng/mL mGM-CSF was added to each well. At days 6 and 8, half of the media was sampled from each well, centrifuged, and the cell pellet was resuspended in 5 mL of fresh RPMI media supplemented with only 10 ng/mL mGM-CSF before re-plating. BMDCs were collected at day 10 (non-adherent cells) and used to evaluate DC activation in normoxia or hypoxia.

In vitro DC activation assay

BMDCs were incubated in complete RPMI medium containing 10 ng/mL mGM-CSF at 37° C. in either humidified 5% CO₂/95% air (normoxia) or 5% CO₂/1% O₂/94% N2 (hypoxia) incubator (Napco CO₂ 1000 hypoxic incubator, ThermoFisher) for 24 h. One cryogel or O₂-cryogel was added to each well prior to starting the incubation. For BMDC activation, the medium was completed with 5 μg/mL CpG ODN 1826. The negative control consisted of BMDCs cultured in complete RPMI medium containing 10 ng/mL mGM-CSF. DC stimulation and maturation was evaluated by flow cytometry using the following fluorescent antibodies (BioLegend): APC-conjugated anti-mouse CD11c (clone N418), PE-conjugated anti-mouse CD86 (Clone GL1), and PerCP/Cyanine5.5-conjugated anti-mouse CD317 (clone 927).

Imaging of encapsulated N and RBD proteins within the cryogel network

RBD or N protein was dissolved in sodium bicarbonate buffer (pH 8.5) at 0.5 mg/mL and reacted with Alexa Fluor 488-NHS ester or Alexa Fluor 647 NHS ester (Click Chemistry Tools), respectively, for 2 h at 4° C. Fluorochrome-modified proteins were purified via spin filtration over 10 kDa Amicon Spin Filters (Sigma Aldrich) and washed 5 times with DPBS. Concentration of purified proteins was determined by UV-Vis absorbance measurements at 280 nm, after correcting for fluorophore absorbance, using the Nanodrop One (ThermoFisher). O₂-cryogels containing the fluorescently labeled RBD and N proteins were fabricated as described above. After thawing, cryogels were washed four times with 1 mL of DPBS and imaged by confocal microscopy (Zeiss 800).

Release of immunomodulatory factors and antigens from cryogels

To determine the in vitro release kinetics of GM-CSF, CpG-ODN, and RBD protein from Cryogel_{VAX} and O₂-Cryogel_{VAX}, gels were briefly washed in 70% ethanol followed by 2 DPBS washes. Each washed gel was incubated in sterile DPBS with 2% BSA in a microcentrifuge tube under orbital shaking at RT. The entire supernatant was removed periodically and replaced with the same amount of fresh buffer. GM-CSF, CpG-ODN, and RBD protein released in the supernatant were detected by either ELISA (GM-CSF: BioLegend ELISA MAX™ Deluxe, RBD: Elabscience SARS-CoV-2 Spike Protein 51 RBD ELISA Kit) or iQuant™ ssDNA quantification assay (GeneCopoeia). The N protein release kinetics was not determined due to the instability of the protein at high concentration, buffer, and study duration.

Antibody titration by enzyme-linked immunosorbent assay (ELISA)

Anti-RBD IgG and IgM antibody titers were determined using a SARS-CoV-2 Spike S1-RBD IgG & IgM ELISA detection kit (Genscript). Anti-N IgG and IgM antibody titers were determined using a SARS-CoV-2 Nucleocapsid Protein IgG ELISA Kit (Lifeome). Both kits were optimized by replacing the HRP-conjugated IgG or IgM anti-human antibody with an HRP-conjugated IgG (H+L) goat anti-mouse antibody (FisherScientific) or an HRP-conjugated IgM (Heavy chain) goat anti-mouse antibody (FisherScientific), respectively. Immunoglobulin isotyping was evaluated using Ig Isotyping Mouse Uncoated ELISA Kit (ThermoFisher) following the manufacturer's recommendation by measuring absorbance at 450 nm on a plate reader (Synergy HT). All ELISAs were performed on mouse sera that were heat-inactivated (30 min at 56° C.). Endpoint titers were determined as the maximum dilution that emitted an optical density exceeding 4 times the background (sera of mice vaccinated with Sham vaccine).

SARS-CoV-2 surrogate virus neutralization test (sVNT)

The detection of neutralizing antibodies against SARS-CoV-2 that block the interaction between RBD and the human ACE2 (hACE2) cell surface receptor was determined using an sVNT according to the manufacturer's protocol (Genscript). Briefly, heat-inactivated mouse sera were pre-incubated with HRP-RBD (30 min at 37° C.) to allow the specific binding of neutralizing antibodies. Then, the mixture was transferred into a plate coated with hACE2 and incubated for 15 min at 37° C. The unbound HRP-RBD, as well as HRP-RBD bound to non-neutralizing antibody, will interact with the hACE2, while neutralizing antibody-HRP-RBD complexes will remain in suspension and will be removed during washing. TMB substrate was used to detect the non-neutralized HRP-RBD. Therefore, the absorbance was inversely proportional to the titer of anti-SARS-CoV-2 neutralizing antibodies. For this experiment, 10-fold dilutions of mouse sera (10⁻¹ to 10⁻⁸) were used.

Cytokine quantification

Cytokine levels in mouse sera and cryogels were quantified using LEGENDplex™ mouse Th cytokine panel (BioLegend) according to the manufacturer's recommendations. Mouse sera were collected at day 24 (3-days post-boost) and diluted 10 and 100 times. Cryogel_{VAX}, O₂-Cryogel_{VAX}, and (blank) cryogels were explanted at day 56, homogenized through a 70 μm cell strainer (FisherScientific), resuspended in 1 mL DPBS, and then centrifuged 5 min at 300 g. The supernatant was collected and diluted 2, 5, and 10 times. The cytokine panel included: IL-2, 4, 5, 6, 9, 10, 13, 17A, 17F, 22, IFNγ and TNFα.

Authentic SARS-CoV-2 plaque reduction neutralization test (PRNT)

Heat inactivated mouse serum samples were serially diluted in DPBS using two-fold dilutions starting at 1:50. Dilutions were prepared in duplicate for each sample and plated in duplicate. Each dilution was incubated in a 5% CO₂ incubator at 37° C. for 1 h with 1000 plaque-forming units/mL (PFU/mL) of SARS-CoV-2 (isolate USA-WA1/2020, BEI). Controls included (1) Dulbecco's Modified Eagle Medium (DMEM, Gibco) containing 2% fetal bovine serum (FBS, Gibco) and 100X antibiotic-antimycotic (Gibco) to a final concentration of 1X as a negative control; and (2) 1000 PFU/mL SARS-CoV-2 incubated with DPBS as a positive control. Each dilution or control (200 μL) was added to two confluent monolayers of NR-596 Vero E6 cells (ATCC) and incubated in a 5% CO₂ incubator at 37° C. for 1 h. A gentle rocking was performed every 15 min to prevent monolayer drying. Cells were then overlaid with a 1:1 solution of 2.5% Avicel® RC-591 microcrystalline cellulose and carboxymethylcellulose sodium (DuPont Nutrition & Biosciences) and 2× Modified Eagle Medium (MEM-Temin's modification, Gibco) supplemented with 100X antibiotic-antimycotic (Gibco) and 100X GlutaMAX (Gibco) both to a final concentration of 2X, and 10% FBS (Gibco). The plates were then incubated in a 5% CO₂ incubator at 37° C. for 2 days. The monolayers were fixed with 10% neutral buffered formalin for at least 6 h (NBF, Sigma-Aldrich) and stained with 0.2% aqueous Gentian Violet (RICCA Chemicals) in 10% NBF for 30 min, followed by rinsing and plaque counting. The half maximal inhibitory concentrations (IC₅₀) were calculated using GraphPad Prism 8 as previously described.

Immune cell characterization in cryogels and LNs

At day 21 and 56, cryogels and LNs were explanted, homogenized over a cell strainer, and single cell suspensions were washed with DPBS. Next, cells were stained with Fixable Viability Dye eFluor 506 (eBioscience, 1:1000 dilution in DPBS) for 30 mins at 4° C. The cells were washed once with DPBS and washed twice with PBA (PBS+1% BSA) before staining of cell surface antigens by overnight incubation of fluorochrome-conjugated antibodies (I-A/I-E-FITC (Clone: M5/114.15.2), CD138-PE (Clone 281-2), CD4-PerCP-Cy5.5 (Clone GK1.5), CD45.2-PE-Cy7 (Clone 104), CD11c-APC (Clone N418), CD8-AF700 (Clone 53-6.7), CD19-APC-Cy7 (Clone 6D5), CD11b-BV421 (Clone: M1/70), CD3-BV605 (Clone 145-2C11), Biolegend) in PBA at 4° C. Cells were washed 3 times with PBA, fixed through incubation in 4% PFA in DPBS for 15 min at 4° C., and washed 3 times with PBA. Flow cytometry measurements were done using the Attune N×T flow cytometer (ThermoFisher).

Splenocyte activation and intracellular cytokine staining

Splenocytes were incubated with 1) 20 ng/mL PMA (Sigma Aldrich) and 1 ug/mL ionomycin (Cell Signaling Technology) (Cell. 2) S protein-derived peptides (GenScript) 3) N protein-derived peptides (GenScript), or 4) control (no stimulation) in presence of 1X Brefeldin A and 1X Monensin (Biolegend) for 6 h at 37° C. After this, the cells were washed with DPBS and incubated for 30 min with Fixable Viability Dye eFluor 780 (eBioscience) in DPBS (1:1000 dilution) at 4° C. After this, cells were washed once with DPBS and washed twice with PBA before staining of cell surface antigens by overnight incubation of fluorochrome-conjugated antibodies (CD3-FITC (Clone 145-2C11), CD4-PerCP-Cy5.5 (Clone GK1.5), CD8-AF700 (Clone 53-6.7), CD44-BV605 (Clone IM7), Biolegend) in PBA at 4° C. Cells were washed 3 times with PBA, after which they were fixed and permeabilized using the Cyto-Fast Fix/Perm Buffer Set (Biolegend) according to the manufacturer's protocol. Intracellular staining was done by incubation of cells with fluorochrome-conjugated antibodies (IL-13-PE (Clone: W17010B), IL-4-PE-Cy7 (Clone: 11B11), IL-17-APC (Clone: TC11-18H10.1), IL-5-BV421 (Clone: TRFKS), IFNγ-BV510 (Clone XMG1.2), Biolegend) in permeabilization buffer for 30 min at 4° C. Cells were washed 3 times with permeabilization buffer, resuspended in PBA, and measured using the Attune N×T flow cytometer (ThermoFisher).

Statistical analysis

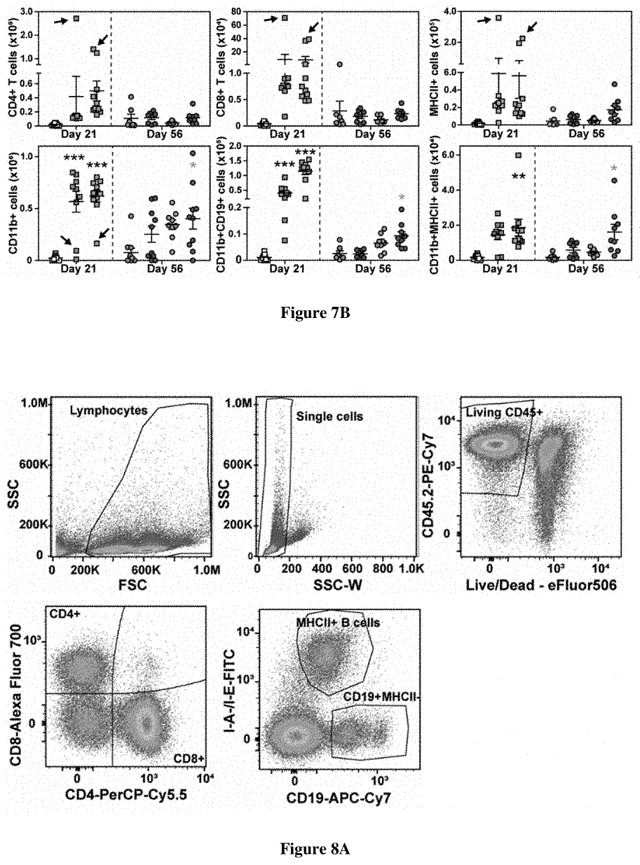

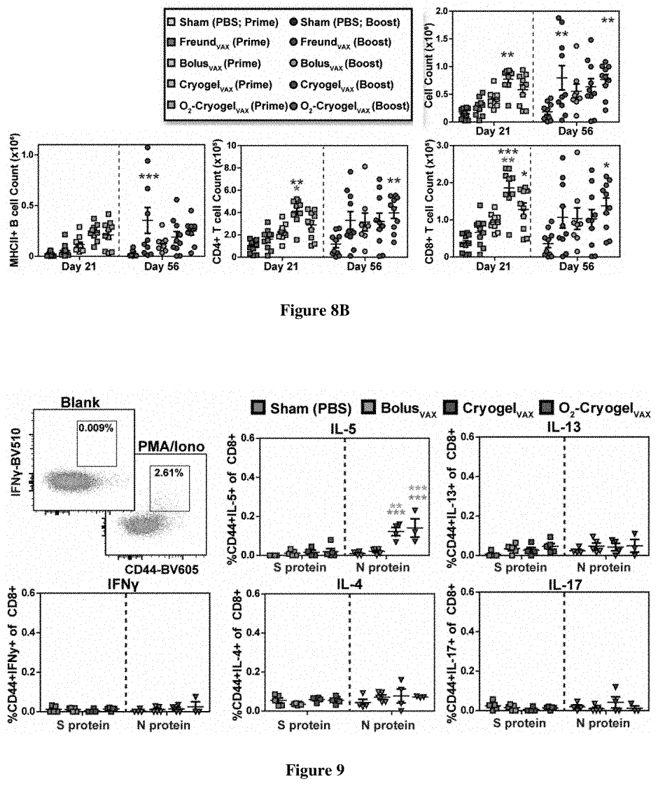

Flow cytometry data were analyzed using FlowJo software. Gating was done as depicted in FIGS. 7A and 8A. Statistical analysis was performed using GraphPad Prism 5 software. Statistical significances were calculated with one-way ANOVA and Bonferroni post-tests to evaluate differences between time points (lines with dark stars indicate statistical differences) or two-way ANOVA and Bonferroni post-tests to evaluate the difference between different conditions/treatments (colored stars indicate statistical differences). P values of 0.05 or less were considered significant. Graphs show the mean ±SEM of calculated values.

Example 2: SARS-CoV-2 vaccine fabrication and characterization

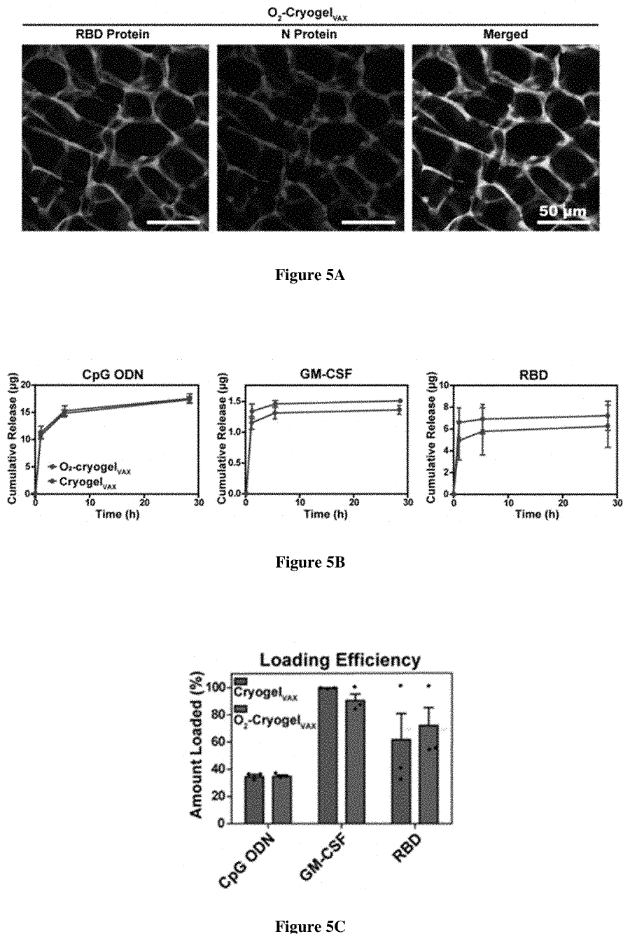

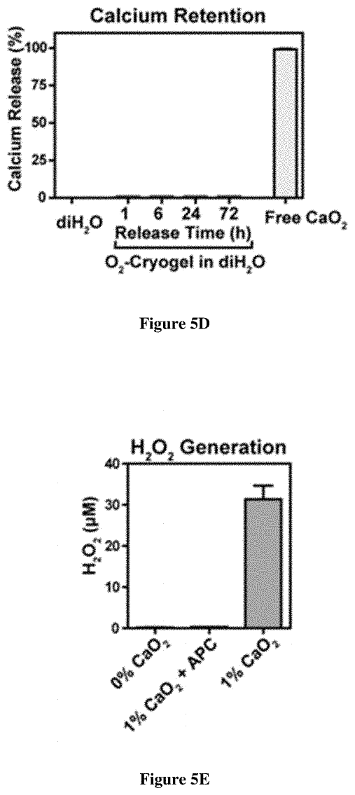

Hyaluronic acid-based cryogel vaccines were fabricated by cryogelation, as previously described (FIG. 1C, steps 1-3). This process results in an elastic biomaterial with a highly interconnected macroporous network, allowing immune cells to traffic in and out of the cryogel. The encapsulation of RBD and N proteins within O₂-Cryogel_{VAX} polymer walls was characterized by confocal microscopy and release from the cryogel by ELISA (FIGS. 5A-5B). Both proteins were effectively entrapped and colocalized within the polymer network. Both Cryogel_{VAX} and O₂-Cryogel_{VAX} exhibited an initial burst release of their payload, followed by a sustained release of the immunomodulatory factors GM-CSF and CpG-ODN 1826 and the antigen RBD (FIG. 5B). Notably, there were no differences among the encapsulation (FIG. 5C) and release profiles of CpG-ODN 1826, GM-CSF, and RBD for the two types of cryogels. Importantly, no significant amounts of Ca²⁺ or H₂O₂ are released from O₂-cryogels (FIGS. 5D-5E), further indicating that they are non-toxic and can safely be used in vivo. These results suggested that cryogels and O₂-cryogels are suitable platforms for controlled vaccine delivery.

Example 3: O₂-Cryogel_{VAX} and Cryogel_{VAX} induce high antibody titers with strong neutralizing activity

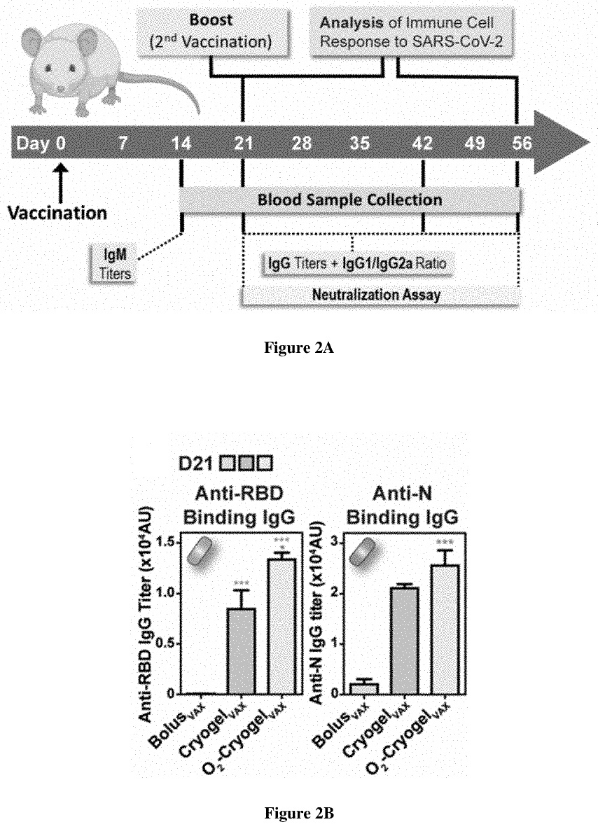

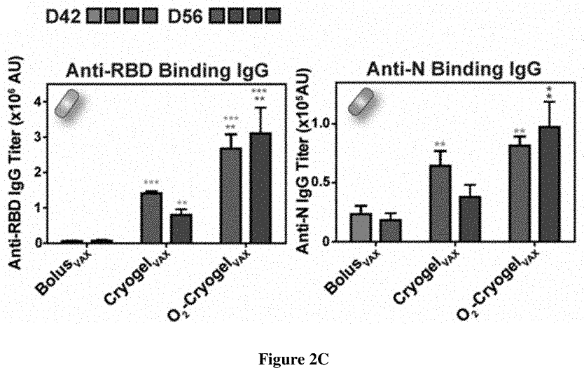

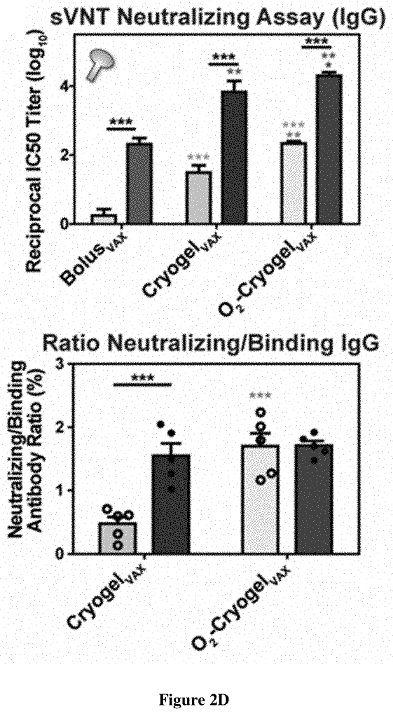

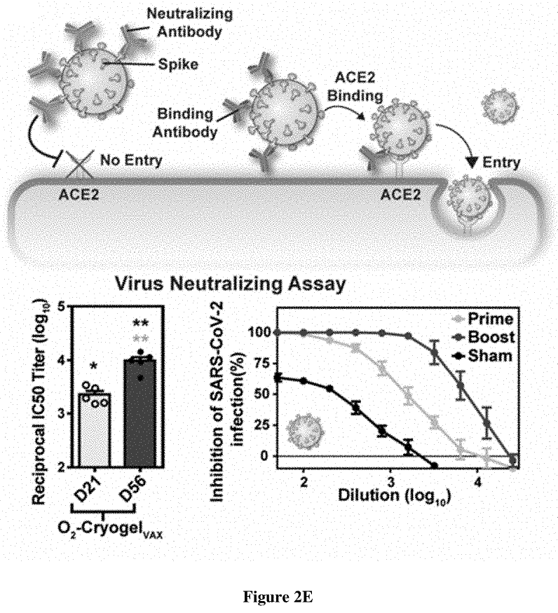

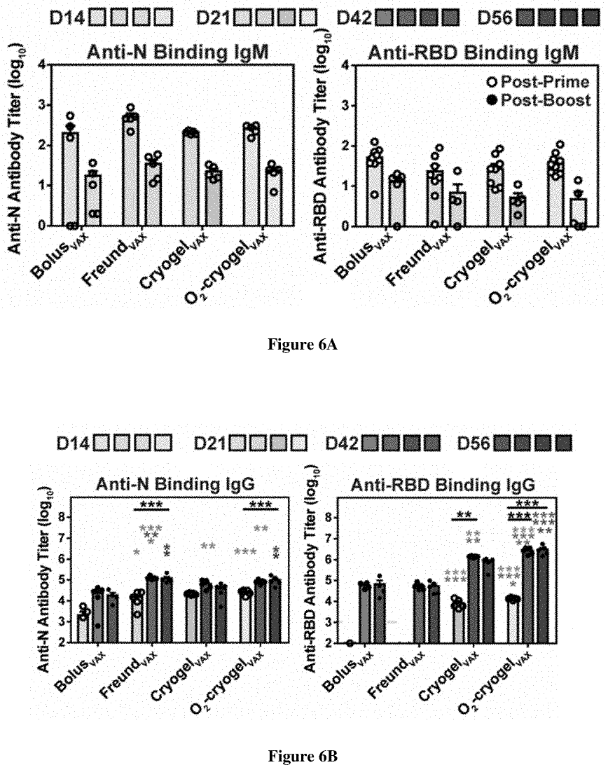

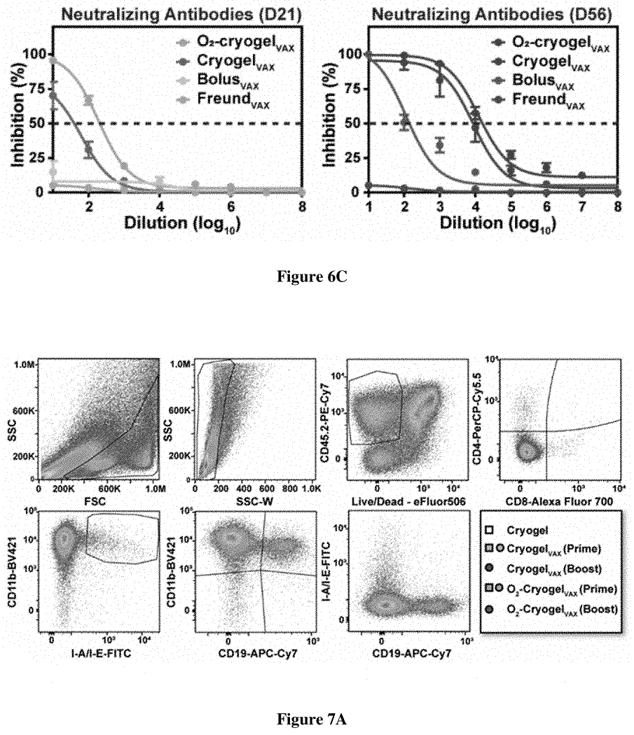

To test the vaccines, eight-week-old female BALB/c mice were immunized by subcutaneous injection of two O₂-Cryogel_{VAX} or Cryogel_{VAX} (one on each flank) at day 0 (prime) and day 21 (boost) (FIG. 2A). Control groups were injected with either PBS (sham-negative control), cryogel-free vaccine (Bolus_{VAX}), or Freund's-based vaccine (Freund_{VAX}-positive control) (Table 1). Blood serum analysis revealed that, although low titers of immunoglobulin M (IgM) antibodies were found across all groups (FIG. 6A), Cryogel_{VAX} and O₂-Cryogel_{VAX} induced high titers of RBD-specific binding immunoglobulin G (IgG) antibodies after only 21 days (FIG. 2B and FIG. 6B). These titers increased substantially following boost immunization, peaking at 1.4×10⁶ at day 42 for animals immunized with Cryogel_{VAX} and 3.1×10⁶ at day 56 for animals immunized with O₂-Cryogel_{VAX}, amounts two orders of magnitude greater than those in control groups (FIG. 2C and FIG. 6B). Interestingly, O₂-Cryogel_{VAX} induced higher production of RBD-specific binding IgG antibodies than Cryogel_{VAX} did, showing a 5-fold increase at day 56, and these titers were sustained for nearly 2 months (study endpoint). Similarly, O₂-Cryogel_{VAX} immunization resulted in high titers of N-specific binding IgG antibodies comparable to those induced by Freund_{VAX}, and 3- and 5-times higher than those generated by Cryogel_{VAX} and Bolus_{VAX}, respectively.

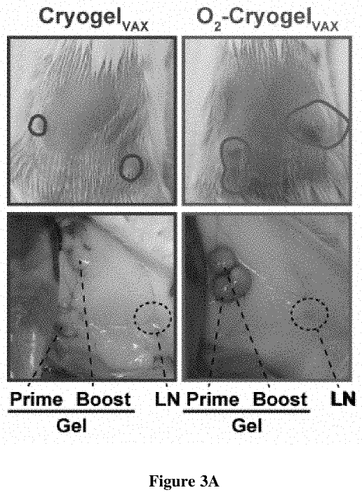

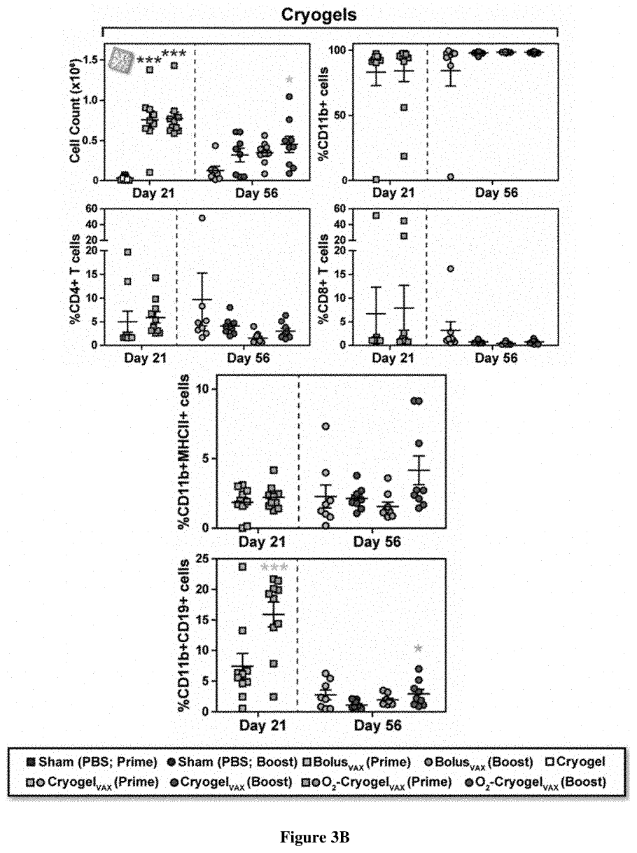

To detect neutralizing antibodies that target the viral spike (S) protein RBD and block its interaction with ACE2, we performed a SARS-CoV-2 surrogate virus neutralization test (sVNT) (FIG. 2D and FIG. 6C). In agreement with the high serological IgG titers, O₂-Cryogel_{VAX} elicited the strongest neutralizing antibody response, with a reciprocal IC₅₀ titer of nearly 20,000 at day 56, which is 3- and 100-fold higher than those from Cryogel_{VAX} and control groups (Bolus_{VAX} and Freund_{VAX}), respectively. Additionally, neutralizing antibodies induced within 3 weeks after only a single immunization with O₂-Cryogel_{VAX} were comparable to those induced after 8 weeks in mice receiving prime and boost vaccinations with Bolus_{VAX} or Freund_{VAX} (FIG. 2D, upper). Importantly, 1.7% of O₂-Cryogel_{VAX}-induced anti-RBD IgG antibodies were neutralizing from day 21 onward (FIG. 3C, lower). We also assessed the neutralization potency of antibodies by plaque reduction neutralization test (PRNT) using VeroE6 cells infected with authentic SARS-CoV-2 (FIG. 2E). As expected, O₂-Cryogel_{VAX} immunization led to high neutralizing titers, which intensified from day 21, reaching a reciprocal IC₅₀ value of nearly 10,000 at day 56 (study endpoint). Collectively, these data demonstrated that the cryogel platform potentiates vaccine efficacy. Furthermore, additive oxygen as a co-adjuvant strongly boosted the humoral response, as shown by the production of antibodies with high neutralizing activity.

TABLE 1
SARS-CoV-2 vaccination groups and dosage

| Group | |
|---|---|
| Sham | 2 × 100 μL PBS |
| Freund_{VAX} | 1 × 100 μL [(25 μg RBD protein + 25 μg N protein + |
| | 1.5 μg GM-CSF − 1:1 ratio with complete Freund's |
| | adjuvant (CFA − Prime) or incomplete Freund's |
| | adjuvant (IFA − Boost)] |
| Bolus_{VAX} | 2 × [(10 μg RBD protein + 10 μg N protein + 1.5 μg |
| | GM-CSF + 50 μg CpG ODN 1826) + 100 μL PBS] |
| Cryogel_{VAX} | 2 × [(10 μg RBD protein + 10 μg N protein + 1.5 μg |
| | GM-CSF + 50 μg CpG ODN1826) + 100 μL PBS] |
| O₂-Cryogel_{VAX} | 2 × [(10 μg RBD protein + 10 μg N protein + 1.5 μg |
| | GM-CSF + 50 μg CpG ODN 1826 + 200 μg of |
| | APC + 200 μg CaO₂) + 100 μL PBS] |

Example 4: O₂-Cryogel_{VAX} promotes local immune cell recruitment and B cell production in LNs

To understand how the vaccines work, we characterized the immune response following prime and prime-boost immunizations in mice. At day 21 and 56, draining LNs, spleens, and cryogels were explanted (FIG. 2A). In comparison to the injection sites of Cryogel_{VAX}, sites of both prime and boost O₂-Cryogel_{VAX} injections were markedly enlarged, indicating increased inflammation and immune cell infiltration (FIG. 3A). Nonetheless, no rash or stress was observed in mice, suggesting that the vaccines were well tolerated. Overall, unlike blank cryogels, large numbers of infiltrated immune cells were retrieved from both types of cryogel vaccines (FIG. 3B and FIGS. 7A, 7B). Most explanted cryogels exhibited low and comparable numbers of CD4+ and CD8+ T cells, whereas high numbers of CD11b-positive myeloid cells, but no DCs, were present (FIG. 3B). Furthermore, the total number of cells positive for the B cell marker CD19 was 2-fold higher in O₂-Cryogel_{VAX} compared to those in blank cryogels (FIG. 7B). However, the exact identity of these cells is unclear, as they were also CD11b-positive and did not have other B cell markers such as MHCII (FIG. 7A). Additionally, only a small population of MHCII-positive CD11b+ cells was observed. Interestingly, evidence for an ongoing adaptive immune response was found in a small number of cryogel vaccines. These cryogel vaccines contained a lower fraction of CD11b+ myeloid cells but relatively greater proportions of T cells and MHCII+B cells (see the outliers in FIG. 3B and FIG. 7B). Compared to day 21, immune cell numbers in prime cryogel vaccines decreased at day 56, indicating that both types of cryogel vaccines do not generate chronic and potentially dangerous inflammatory responses.

Analysis of LNs in mice immunized with Cryogel_{VAX} and O₂-Cryogel_{VAX} confirmed that a robust immune response was induced. This resulted in at least a 4-fold greater increase in total immune cell numbers than that observed among mice receiving sham injections at both time points (FIG. 3C and FIGS. 8A, 8B). In particular, the frequency of MHCII+B cells within LNs was greatly increased in mice immunized with both cryogel-based vaccines. Although the frequency of CD4+ T cells was reduced at day 21 in LNs from mice receiving cryogel vaccines (FIG. 3C), overall CD4+and CD8+ T cell numbers increased after vaccination (FIG. 8B). These data showed that cryogel vaccines induce a strong B cell-mediated immune response in LNs and display restrained adaptive immune responses within the cryogels following initial priming.

Example 5: O₂-Cryogel_{VAX} enhances both Th1- and Th2-associated immune responses

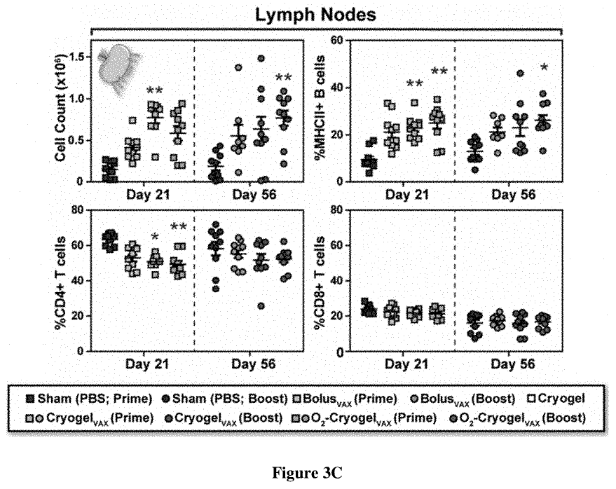

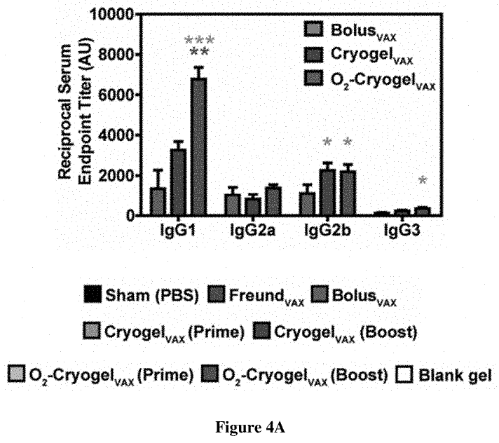

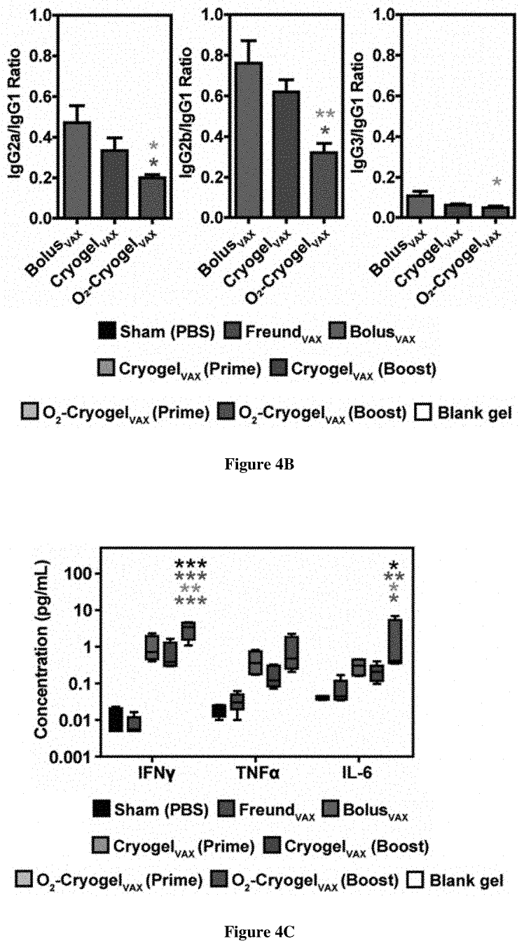

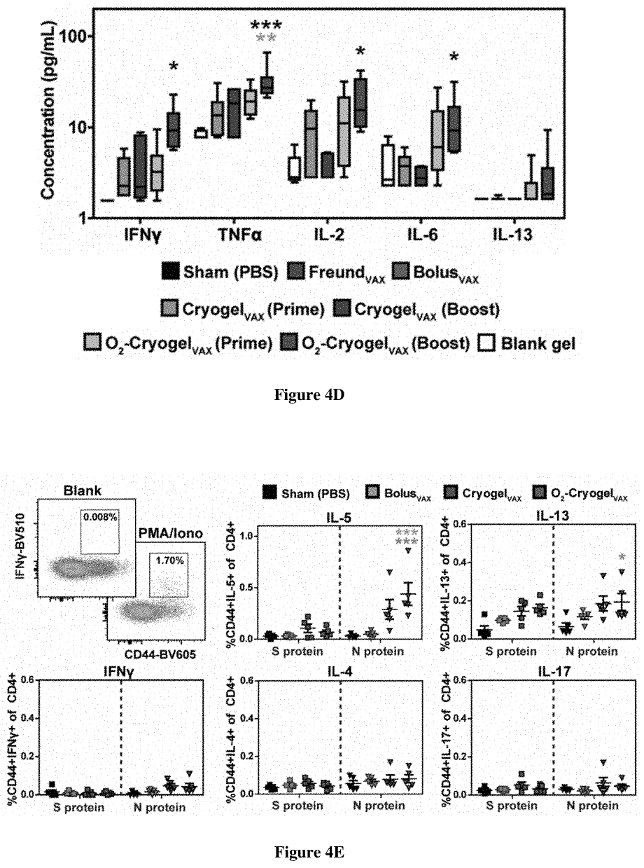

Next, to evaluate the immunostimulatory effects of cryogel-based SARS-CoV-2 vaccines, we analyzed the balance between Th1 and Th2 immune responses. Production of antibody subclass IgG1 is indicative of Th2 responses, and IgG2a/b/c and IgG3 are indicative of Th1 responses. In our study, vaccines across all groups elicited IgG2 and IgG1 subclass RBD-binding antibodies, indicating induction of both Th1 and Th2 immune responses (FIG. 4A). Both cryogel-based vaccines promoted the production of IgG2b. However, O₂-Cryogel_{VAX} improved IgG1 production resulting in lower IgG2a/IgG1 and IgG2b/IgG1 ratios (FIG. 4B). Interestingly, O₂-Cryogel_{VAX} was the only vaccine that induced IgG3 production (FIG. 4A-B).

Th1 and Th2 responses are also associated with different cytokine profiles: interferon y (IFNγ), tumor necrosis factor a (TNFα), and interleukin-2 (IL-2) indicate Th1 responses; IL-4, IL-5, and IL-13 indicate Th2 responses. Thus, we quantified cytokines in serum at day 24 (FIG. 5C) and in explanted cryogels at day 56 (FIG. 4D). At day 24, all vaccines induced detectable concentrations of pro-inflammatory interleukin-6 (IL-6) and Th1 cytokines (IFNγ and TNFα) in mouse sera (FIG. 4C). Interestingly, concentrations of IFNγ and IL-6 in mice immunized with O₂-Cryogel_{VAX} were 3- or 10-fold higher than their concentrations in mice immunized with Bolus_{VAX} or Cryogel_{VAX}, respectively. Similar results were observed at day 56 (FIG. 4D). Higher concentrations of Th1 cytokines IFNγ, TNFα, and IL-2, as well as IL-6, were quantified in mice immunized with O₂-Cryogel_{VAX}, compared to those immunized with Cryogel_{VAX}. Furthermore, we noted low concentrations of the Th2 cytokine IL-13 in O₂-Cryogel_{VAX}. As expected, blank cryogels were associated with low or negligible amounts of these cytokines.

To more directly assess the Th1/Th2 immune responses, we investigated the cytokine profile of antigen-specific T cells generated with both cryogel vaccines. The intracellular production of cytokines by splenocytes from immunized mice was examined following stimulation with peptides derived from viral S or N proteins. Cells were isolated at day 21 after prime immunization. Splenocytes from O₂-Cryogel_{VAX}-immunized mice stimulated with N-derived peptides showed increased fractions of IL-5-producing CD4+ and CD8+ T cells and IL-13-producing CD4+ T cells (FIG. 4E and FIG. 9). These results indicated the presence of N protein-specific Th2 cells. However, no differences were noted following stimulation with S-derived peptides, and the proportions of IFNγ-, IL-4-, or IL-17-producing T cells were also comparably low. Collectively, these data suggested that both types of cryogel vaccines elicited balanced Th1/Th2 immune responses, even though it was more prominent for O₂-Cryogel_{VAX}.

Example 6: O₂-cryogels enable controlled release of oxygen in their surrounding environment

Kinetics of oxygen release were determined using contactless optical oxygen microprobes (PyroScience GmbH, Aachen, Germany). Briefly, square-shaped cryogels, O₂-cryogels, and APC-free O₂-cryogels were individually placed into a 96 well plate containing 200 μL of PBS and incubated at 37° C. under normoxic conditions using a Napco CO₂ 1000 incubator (Thermo Fisher Scientific). The microprobe was centered in the bottom of the wells, and dissolved oxygen (μmol/L, 1 point every 300 seconds) within the surrounding environment of cryogels were recorded for 24 h.

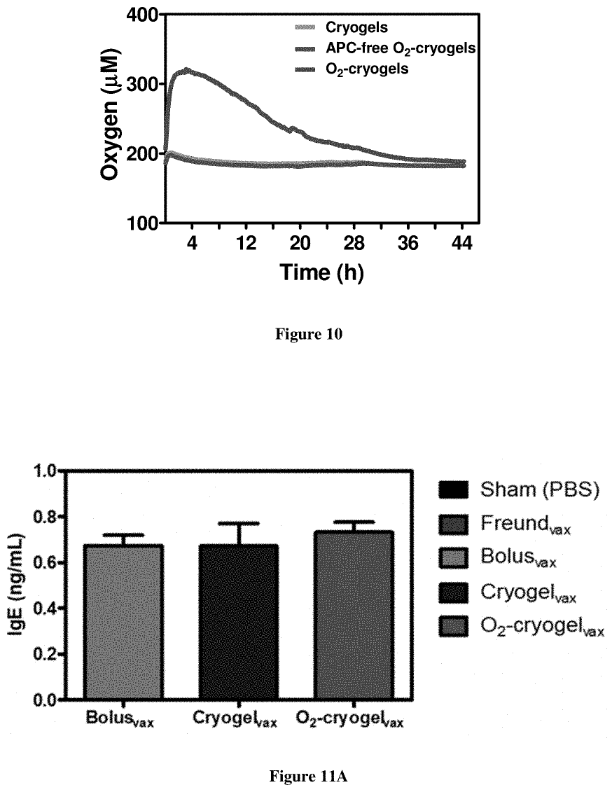

The capacity of O₂-cryogels to reduce local hypoxia was first evaluated. O₂-cryogels and cryogels were fabricated as previously described, and their ability to generate oxygen in vitro was confirmed using contactless environmental oxygen probes (FIG. 10). As intended, O₂-cryogels released oxygen in their surrounding environment for nearly 48 h, with oxygen level peaking at 310 μM after 3 h. On the contrary, catalase-free O₂-cryogels produced an insignificant amount of oxygen, confirming the need to use this enzyme.

Example 7: Immunization with O₂-Cryogel_{VAX} does not trigger an allergic response

Immunoglobulin isotyping was evaluated using Ig Isotyping Mouse Uncoated ELISA Kit (Thermo Fisher Scientific) and IgE Mouse ELISA kit (Thermo Fisher Scientific) following the manufacturer's recommendation by measuring absorbance at 450 nm on a plate reader (Synergy HT).

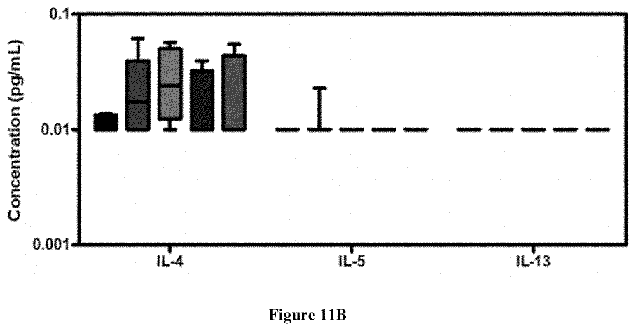

Vaccines do not elicit allergic reactions (FIG. 11A). No vaccine-driven allergic inflammation was observed in mice as supported by the low or negligible levels of serum IgE antibodies (FIG. 11A) and cytokines (FIG. 11B) involved in allergic reactions (IL-4, IL-5, and IL-13).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A vaccine, comprising an oxygen-generating cryogel, wherein the oxygen-generating cryogel comprises an antigen, a chemoattractant/stimulating factor for immune cells, an adjuvant, and an oxygen-producing compound.

2. The vaccine of claim 1, wherein the antigen is a polysaccharide, a lipid, or a nucleic acid.

3. (canceled)

4. The vaccine of claim 2, wherein the antigen is derived from a pathogen.

5-6. (canceled)

7. The vaccine of claim 4, wherein the pathogen is a virus.

8. The vaccine of claim 7, wherein the virus is a coronavirus, varicella-zoster virus, hepatovirus A, hepatitis B virus, hepatitis C virus, human papillomavirus, influenza virus, measles virus, marburg virus, rabies lyssavirus, variola virus, dengue virus, hantavirus, ebola virus, human papillomavirus, mumps virus, rubella virus, poliovirus, or rotavirus.

9. The vaccine of claim 8, wherein the virus is a coronavirus.

10. The vaccine of claim 9, wherein the coronavirus is selected from SARS-CoV, MERS-CoV, HCoV, HKU1, and SARS-CoV-2.

11. The method of claim 10 wherein the coronavirus is SARS-CoV-2.

12-13. (canceled)

14. The vaccine of claim 4, wherein the pathogen is a bacterium, fungi, protozoa, worms, parasites, or an infectious protein.

15-18. (canceled)

19. The vaccine of claim 1, wherein the chemoattractant is granulocyte macrophage colony stimulating factor (GM-CSF), Flt3L, CCL-19, CCL-20, CCL-21, N-formyl peptide, ffactalkine, monocyte chemotactic protein-1, and MIP-3a.

20. The vaccine of claim 1, wherein the adjuvant is a toll-like receptor 9 (TLR9) ligand.

21. The vaccine of claim 20, wherein the TLR9 ligand comprises a cytosine-guanosine oligonucleotide (CpG-ODN).

22-24. (canceled)

25. The vaccine of claim 1, wherein the adjuvant is a lipopeptide, glycolipid, proteolipid, virus, lipopolysaccharide, protein, mycoplasma, bacteria, fungi, peptide, polysaccharide, small synthetic molecule, toxoplasma gondii, oil, or inorganic molecule.

26-35. (canceled)

36. The vaccine of claim 1, wherein the oxygen-producing compound is a peroxide, an oxide, a percarbonate or a fluorinated compound.

37. The vaccine of claim 1, wherein the oxygen-producing compound is CaO₂, (Na₂CO₃)₂,1.5H₂O₂, MgO₂, encapsulated H₂O₂/Polyvinylpyrrolidone, magnesium peroxide, hydrogen peroxide, manganese dioxide, perfluorochemicals, zinc oxide, or sodium percarbonate.

38. The vaccine of claim 1, wherein the oxygen-generating cryogel further comprises a catalase.

39-69. (canceled)

70. A method of modulating the immune system, comprising administering to a subject in need thereof an effective amount of the vaccine of claim 1, thereby modulating the immune system in the subject.

71. A method of stimulating the immune system, comprising administering to a subject in need thereof an effective amount of the vaccine of claim 1, thereby stimulating the immune system in the subject.

72. A method of inducing an immune response, comprising administering to a subject in need thereof an effective amount of the vaccine of claim 1, thereby inducing an immune response in the subject.

73. A method of preventing an infection caused by a pathogen, comprising administering to a subject in need thereof an effective amount of the vaccine of claim 1.

74-87. (canceled)
